(19) United States
(12) Patent Application Publication
(10) Pub. No.: US 2023/0390090 A1
(43) Pub. Date: Dec. 7, 2023

(54) NITINOL STENTS AND METHODS OF FABRICATION THEREOF

(71) Applicant: National University of Singapore, Singapore (SG)

(72) Inventors: Sin Liang SOH, Singapore (SG); Lina YAN, Singapore (SG); Ying Hsi Jerry FUH, Singapore (SG)

(21) Appl. No.: 18/040,783

(22) PCT Filed: Aug. 5, 2021
(86) PCT No.: PCT/SG2021/050460

§ 371 Date: Feb. 6, 2023

(30) Foreign Application Priority Data
Aug. 6, 2020 (SG) 10202007537P

(51) Int. Cl. A61F 2/915; A61F 2/966; B22F 5/10; B22F 10/28; B22F 10/366; B22F 10/368

(52) U.S. Cl. CPC A61F 2/915; A61F 2/966; B22F 5/106; B22F 10/28; B22F 10/366; B22F 10/368; A61F 2002/91575; B33Y 10/00

(57) ABSTRACT

The present disclosure relates to a method of 3D printing a stent, comprising performing selective laser melting on a Nitinol powder in order to form the stent, wherein selective laser melting is performed with particular parameters. The 3D printed stent can be curved. The present disclosure also relates to the 3D printed stent thereof, a stent delivery device comprising a tube and a crimped 3D printed stent slidably disposed within the tube, and a method of delivering a stent in a stent delivery device into a channel.

### TECHNICAL FIELD

The present invention relates, in general terms, to stents formed from Nitinol. The present invention also relates to methods of 3D printing Nitinol stents.

### BACKGROUND

Stents are small tube-like surgical devices used by surgeons to unblock or widen clogged arteries to restore regular blood flow for treatment of patients with vascular diseases. Traditionally, stents are made of a biocompatible Stainless Steel or metal alloy.

Stent sizing and apposition have been shown to be important determinants of clinical outcome. Undersized stents tend to induce thrombosis in the longer term whereas oversizing increases the vessel wall stress and may induce inflammatory response, which, in turn, contributes to neointimal hyperplasia. Failure to achieve predicted stent diameter is a common problem for stents made of stainless steel and cobalt chrome when deploy using semi-compliant balloons. Therefore, selection of proper stent size relative to the target vessel should be considered as important as post-deployment optimization strategy.

In the pre-stent era restenosis ranged between 32-55% of all angioplasties and drop to 17-41% in the bare metal stent (BMS) era. A further step to reduce restenosis was undertaken with the introduction of drug-eluting stents (DES), with a reduction to numbers <10%. DES are used to counter In-Stent Restenosis by improving blood flow and decrease the likelihood of repeating procedures to reopen blocked blood vessels compared to uncoated devices. However, in recent times, the U.S Food and Drug Administration (FDA) has expressed concerns on whether Paclitaxel-coated balloons and stents have a long-term adverse effect such as late mortality. Drugs use serve as a preventive solution not rectifying the root cause of In-stent restenosis and stent thrombosis which is mainly due to vascular injuries caused by over-expanded stents over straining the vascular walls.

Although polymer bioresorbable stents have previously been made, polymer-based scaffolds display inferior mechanical performance compared with metallic drug eluting stents (DES). Polymeric surfaces often offer less than ideal conditions for endothelial cell migration, thrombogenic resistance, inflammation, and vessel wall healing.

Other challenges remain. For example, failure of vessel wall healing has been attributed either to the use of polymeric stent coatings, or due to the effects of the eluted drug.

Hemodynamics is the dynamics of blood flow, and in medical contexts it often refers to basic measures of cardiovascular function, such as arterial pressure or cardiac output. Stents, which are deployed to reopen stenotic regions of arteries and to restore blood flow, have risks of causing inflammation and localised stent thrombosis that would result in a stent failure. Stent edge restenosis, the formation of a neointima that gradually re-narrows the arterial lumen, is recurrent in 30-40% of patients receiving BMS. Even though there are more advanced DES that release anti-proliferative drugs to successfully address the problem of restenosis, DES recipients are still significantly associated to late-stent thrombosis (LST).

Stent thrombosis is a thrombotic occlusion of a coronary stent, which is the formation of blood clot inside the blood vessels, resulting in the obstruction of blood flow, and it is an acute process in contrast to restenosis. Studies showed strong correlations between LST and the lack of endothelial coverage. The endothelium is a single layer of endothelial cells lining the vascular walls and plays an integral part in maintaining vascular homeostasis. Stenting causes significant damage to the vascular wall and endothelium, resulting in inflammation, repair and the development of neointimal hyperplasia. The ability of the endothelial to repair itself is dependent on the migration of neighbouring mature endothelial cells and the attraction of circulating endothelial progenitor cells to the injured area, which then differentiate into endothelial-like cells.

Endothelialization of the stent's strut surface is inversely proportional to the thickness of the stent and areas with largest flow separation zone. The shape of individual struts promotes blood flow separation that creates recirculation zones, and slower flow in a recirculation zone yields lower shear rates that retards endothelialization. Recirculation zones can also serve as micro-reaction chambers where procoagulant and pro-inflammatory elements from the blood and vessel wall accumulate.

Accordingly, there is a need to develop new stent designs. There is also a need to develop stents using other materials which are more suitable.

Stents can be fabricated with various techniques, such as etching, micro-electro discharge machining, electro-forming and die-casting. In the market currently, most stents are fabricated using laser cutting technology such as micro-laser machining technology. The process involves a high energy density laser beam focusing on the workpiece surface, where the thermal energy that is absorbed heats and transforms the workpiece volume into a molten, vaporized or chemically changed state that can be easily removed by the flow of a high pressure assist gas jet.

However, laser cutting affects the quality issue of the workpiece due to long pulse duration and melting. This long pulse machining results in heat affected zone (HAZ) generation at the vicinity of the cut regions, as well as significant slog, dross, recast and oxide layer, and back wall damage that will require post-processing. Advancement of the laser industry has driven the development of shorter pulse lasers that would offers opportunities to minimise and eliminate HAZ. However, fabricating stents through laser cutting still requires system optimisation to achieve high yield, high quality and low cost.

There has been recent interest in using Nitinol for making stents. Nitinol is a metal alloy of Nickel and Titanium, where the two elements are present in roughly equal atomic percentages. It exhibits shape memory, superelasticity, good corrosion resistance and good biocompatibility. As such, it is highly sought after for medical applications. The global market for Nitinol-based medical devices is expected to grow at a compound annual growth rate of nearly 8.2% from 2017 to 2025. Reason for the rising global Nitinol medical devices market is the prevalence of cardiovascular diseases, growing population susceptible to peripheral artery diseases and increasing demand for minimally invasive surgical procedures. As per statistics of the World Health Organization, almost 17.7 million individuals suffer from cardiovascular diseases each year; cardiovascular diseases account for 31% deaths each year globally. Advantageously, the treatment of iliac artery occlusive disease with self-expandable stent as compared with Balloon expanded stent resulted in a lower 12-month restenosis rate and a significantly reduced TLR rate.

However, much difficulty is faced with processing Nitinol structures which are sufficiently thin for use in biomedical application such as stenting. Some reasons include surface quality, mechanical properties and biocompatibility. Partially unsintered powder, especially Nickel, could remain on the stent surface or be released into the bloodstream, which could have an adverse biological effect.

Additionally, as laser machining employs a high energy laser beam to precisely heat, melt and vaporise a tube of Nitinol material, heat affected zones are created. Not only does this create surface defects such as burrs and dross formation, but it also affects the microstructure.

While an ultrashort pulse laser can produce a dross-free cut of Nitinol, ultrashort pulse laser machining processes have low cutting efficiency. In addition, debris and recast formation from the vaporized material is still required to be removed by other methods.

Moreover, the tube-based cross section patterns and non-streamlined laser-moving paths in laser-machining have further reduced the design freedom of stents, raising the risk of stents thrombosis originated from blood flow separation.

Accordingly, there is a need to develop new technologies to produce stents. Even when a new technology is used to produce stents, there are concerns that the stents will not perform as expected due to the change in fabrication process.

It would be desirable to overcome or ameliorate at least one of the above-described problems, or at least to provide a useful alternative.

### SUMMARY

The present invention is predicated on the understanding that additive manufacturing (AM) can be a more economical solution to fabricate a high cost stent. In this method, instead of using high heat energy to vaporise the material, a laser is used as a heat source to precisely fuse metal powder particles together, layer by layer. In particular, while selective laser melting (SLM) has been used for making structures from metals such as steels, aluminium and titanium, a stable process for Nitinol has yet to be established. There is also a considerable mismatch in the fabrication parameters with feature sizes of earlier reported SLM processed nitinol stents. There is also a need to develop methodologies for creating features of about 100-300 μm, or wires with a diameter of less than 1 cm, or preferentially less than 0.5 mm. Without wanting to be bound by theory, the inventors have found that stents having wires with less than 1 cm can be reliably printed with superelastic properties and/or shape memory properties by controlling at least the laser power and scanning speed when printing. Further, additional considerations (such as boundary, downskin and upskin processing, support fabrication, and special geometries, e.g., thin-walled constructs) need to be taken into account to create a stent that has Nitinol's inherent unique properties of shape memory effect and superelasticity, as well as extrinsic minimal surface roughness and porosity, apart from the basic mechanical properties. With proper selection of process parameters, heat affected zones can be eliminated or minimized. To this end, SLM offers great potential for producing Nitinol stents.

Additionally, stents fabricated by AM have the capability to deliver customized parts which may not be feasible and cost effective using conventional manufacturing methods. This is favourable for medical applications in which patient-specific implant design can be realised to ensure a better anatomically fit thus promotes faster healing.

The present invention provides a method of 3D printing a stent, comprising: performing selective laser melting on a Nitinol powder in order to form the stent, wherein selective laser melting is performed with:
i) a laser power of about 50 W to about 150 W, and a scanning speed of about 50 mm/s to about 1000 mm/s; or
ii) a laser power of about 150 W to about 250 W, and a scanning speed of about 500 mm/s to about 3000 mm/s; or
iii) a laser power of about 150 W to about 250 W, and a scanning speed of about 50 mm/s to about 500 mm/s; or
iv) a laser power of about 250 W to about 350 W, and a scanning speed of about 500 mm/s to about 3000 mm/s.

It was found that when 3D printing using these conditions, the resultant stents have shape memory and/or superelastic properties. In particular, the 3D printed stent can be customised such that it is curved and which retains its curved configuration due to the shape memory property. The 3D printed stent can be crimped into a linear configuration and inserted into a delivery tube for deployment at a target site. When deployed at about human body temperature, the crimped stent reverts back to its original printed size and shape, and is further superelastic in the sense that it can maintain its size and shape after removal of external forces such as muscle contractions. In contrast, conventional stents are produced by laser cutting, and can only be made with superelastic properties. Conventional stents are also made with a linear configuration due to the difficulties in producing curved stents.

In some embodiments, the selective laser melting is performed with:
i) a laser power of about 50 W to about 150 W, and a scanning speed of about 50 mm/s to about 500 mm/s; or
ii) a laser power of about 150 W to about 250 W, and a scanning speed of about 500 mm/s to about 1500 mm/s; or
iii) a laser power of about 150 W to about 250 W, and a scanning speed of about 50 mm/s to about 150 mm/s; or
iv) a laser power of about 250 W to about 350 W, and a scanning speed of about 500 mm/s to about 1500 mm/s.

In some embodiments, when the selective laser melting is performed using conditions in (i), the 3D printed stent is characterised by a Aₛ temperature of about −45° C. to about −25° C.

In some embodiments, when the selective laser melting is performed using conditions in (i), the 3D printed stent is characterised by a Mₛ temperature of about −10° C. to about 10° C.

In some embodiments, when the selective laser melting is performed using conditions in (ii), the 3D printed stent is characterised by a Aₛ temperature of about −30° C. to about 0° C.

In some embodiments, when the selective laser melting is performed using conditions in (ii), the 3D printed stent is characterised by a Mₛ temperature of about −10° C. to about 25° C.

In some embodiments, when the selective laser melting is performed using conditions in (i) or (ii), the 3D printed stent is characterised by columnar grains due to inter-layer over melt.

In some embodiments, when the selective laser melting is performed using conditions in (iii), the 3D printed stent is characterised by a Aₛ temperature of about 10° C. to about 75° C.

In some embodiments, when the selective laser melting is performed using conditions in (iii), the 3D printed stent is characterised by a Mₛ temperature of about 60° C. to about 100° C.

In some embodiments, when the selective laser melting is performed using conditions in (iv), the 3D printed stent is characterised by a Aₛ temperature of about 10° C. to about 80° C.

In some embodiments, when the selective laser melting is performed using conditions in (iv), the 3D printed stent is characterised by a Mₛ temperature of about 20° C. to about 70° C.

In some embodiments, when the selective laser melting is performed using conditions in (iii) or (iv), the 3D printed stent is characterised by fully merged layer boundaries due to re-melt of an underlying layer.

In some embodiments, the selective laser melting is performed with a hatch distance of about 0.1 mm to about 0.5 mm.

In some embodiments, the selective laser melting is performed with a layer thickness of about 0.01 mm to about 1 mm.

In some embodiments, the 3D printed stent has a wire diameter of less than 1 cm, preferably less than 0.5 mm.

In some embodiments, the method further comprises a step of heat treating the stent.

In some embodiments, the method further comprises a step of heat treating the stent when the stent is printed using condition ii, iii or iv.

Conventionally, stents fabricated using laser cutting require a heat treatment process called shape setting to obtain its superelastic property. Using selective laser melting and particularly the above parameters, it is not necessary for the stents to undergo heat treatment to achieve the desired properties. Rather, heat treatment may be used to further fine-tune the mechanical properties of the 3D printed stents. For example, the heat treating step causes the distribution of nickel and titanium within the stent to be re-distributed. This lowers the Mₛ temperature, thus allows for a transition from a crimped state to an original uncrimped state at or near human body temperature.

In some embodiments, the heat treating step comprises heating the stent from about 200° C. to about 800° C.

In some embodiments, the 3D printed stent is characterised by a austenite finish temperature (A_{f}) of about 25° C. to about 50° C.

In some embodiments, the 3D printed stent is characterised by wires of the 3D printed stent having a partially flat cross sectional shape.

In some embodiments, the 3D printed stent is characterised by wires of the 3D printed stent having an elliptical, tear drop, partially flattened tear drop or circular cross section shape.

In some embodiments, the 3D printed stent is characterised by a curvature along its longitudinal dimension when in the expanded state.

In some embodiments, the 3D printed stent is characterised by a curvature of about 1° to about 160°.

In some embodiments, the 3D printed stent is characterised by a radius of curvature of about 1 mm to about 200 cm.

In some embodiments, the method further comprises providing a template of the stent; wherein the stent template comprises:
i) at least two circumferential sections that are radially expandable in order for the stent to move from a collapsed state to an expanded state; and
ii) one or more flex sections, each flex section extending between two adjacent circumferential sections, each flex section being longitudinally expandable in order for the stent to move from the collapsed state to the expanded state;
wherein each flex section comprises a plurality of circumferentially arranged flex units, each flex unit comprising a wire having a wave-like structure; and
wherein in the expanded state, the flex unit forms an angle of about 15° to about 90° relative to a local radial plane at a junction with each of the adjacent circumferential sections.

In some embodiments, the wave-like structure is a sinusoidal wave-like structure or a helical wave-like structure.

In some embodiments, each flex unit has a wave number of about 0.5 unit to about 2 units.

In some embodiments, the wave-like structure in each flex unit has a peak characterised by an angle of about 15° to about 90° relative to a local radial plane at the peak.

In some embodiments, when in the expanded state, each flex unit has a transverse breadth of about 2 mm to about 12 mm.

In some embodiments, when in the expanded state, each flex unit has a longitudinal length of about 5 mm to about 15 mm.

In some embodiments, a first end of at least one flex unit is connected to one of two adjacent circumferential sections by a first extension, and/or a second end of at least one flex unit is connected to the other of the two adjacent circumferential sections by a second extension.

In some embodiments, the first extension has a length of about 0.1 mm to about 5 mm and/or the second extension has a length of about 0.1 mm to about 5 mm.

The present invention also provides a 3D printed stent printed using the method as disclosed herein, the stent comprising Nitinol having a nickel content of about 54 wt % to about 57 wt % of the composition and a titanium content of about 43 wt % to about 46 wt % of the composition;
wherein the stent has a martensite to austenite transition (Aₛ) temperature of about −° C. to about 80° C.; and
wherein the stent has a austenite to martensite transition (Mₛ) temperature of about −10° C. to about 100° C.

In some embodiments, when the stent has a Aₛ temperature of about −45° C. to about 0° C. and a Mₛ temperature of about −10° C. to about 25° C., the stent is characterised by columnar grains due to inter-layer over melt.

In some embodiments, when the stent has a Aₛ temperature of about 10° C. to about 80° C. and a Mₛ temperature of about 20° C. to about 100° C., the stent is characterised by fully merged layer boundaries due to re-melt of an underlying layer.

In some embodiments, the 3D printed stent is characterised by a austenite finish temperature (A_{f}) of about 25° C. to about 50° C.

In some embodiments, the nickel is about 54.5 wt % to about 55.8 wt % the composition.

In some embodiments, the nickel is about 55.2 wt % of the composition.

The present invention also provides a stent delivery device, comprising:
a) a tube; and
b) a crimped stent slidably disposed within the tube, the crimped stent comprising Nitinol having a nickel content of about 54 wt % to about 57 wt % of the composition and a titanium content of about 43 wt % to about 46 wt % of the composition;
wherein the crimped stent has a martensite to austenite transition (As) temperature of about −45° C. to about 80° C.; and wherein the crimped stent has a austenite to martensite transition (Ms) temperature of 30 about −10° C. to about 100° C.;
wherein the crimped stent is adapted to revert back to its original uncrimped state when ejected from the tube and when exposed to a temperature of about 25° C. to about 50° C.

As the crimped stent has shape memory properties, the stent is revertible back to its original uncrimped state when at least exposed to a temperature above its Aₛ temperature.

In some embodiments, the stent in its original uncrimped state is adapted to revert back to its original configuration after release of an external force and when exposed to a temperature of about 25° C. to about 50° C.

In some embodiments, the stent delivery device further comprises ejecting means for ejecting the crimped stent out from the tube.

The present invention also provides a method of delivering a stent in a stent delivery device into a channel, the stent delivery device comprising:
a) a tube; and
b) a crimped stent slidably disposed within the tube, the crimped stent comprising Nitinol having a nickel content of about 54 wt % to about 57 wt % of the composition and a titanium content of about 43 wt % to about 46 wt % of the composition;
wherein the crimped stent has a martensite to austenite transition (As) temperature of about −45° C. to about 80° C.; and
wherein the crimped stent has a austenite to martensite transition (Ms) temperature of about −10° C. to about 100° C.;
wherein the crimped stent is adapted to revert back to its original uncrimped state when ejected from the tube and when exposed to a temperature of about 25° C. to about 50° C.,
   the method comprising:
i) ejecting the crimped stent from the stent delivery device into the channel; and
ii) exposing the crimped stent to a temperature of about 25° C. to about 50° C. in order to revert the crimped stent to its original uncrimped state.

### BRIEF DESCRIPTION OF THE DRAWINGS

Embodiments of the present invention will now be described, by way of non-limiting example, with reference to the drawings in which:

FIG. 1 is a schematic diagram of examples of closed-cell and open-cell stents;

FIG. 2 is a schematic of a stent design with excess overhang;

FIG. 3 illustrates some examples of known stents;

FIG. 4A-C illustrates a stent according to embodiments of the present invention, D illustrates corresponding schematics defining a downskin angle;

FIG. 5A-B illustrates simulation results of stress concentrations and displacement in flex units;

FIG. 6A-B illustrates a stent according to certain embodiments;

FIG. 7A-B illustrates stents according to other embodiments of the present invention;

FIG. 8 illustrates cross-sections of a wire forming the stent;

FIG. 9A-B are examples of curved stents according to embodiments of the present invention;

FIG. 10A-B plots the distribution of printing parameters characterised by different regimes;

FIG. 11 illustrates the density of stents examined under High resolution X-ray Computed Tomography (HRXCT);

FIG. 12 shows surfaces of wires fabricated using additive manufacturing; and

FIG. 13 illustrates the change in the stent when subjected to body temperature (about 37° C.);

FIG. 14 illustrates a crimping and re-deployment process of a stent with curved profile;

FIG. 15 shows simulation results of a stent with straight profile deployed in curved vessel and comparison of a commercial stent with a stent of the present invention;

FIG. 16A shows the martensitic transformation starting temperatures (Ms) of nitinol stents printed using different laser power and scanning speed;

FIG. 16B shows a process window for the Ms temperature distribution;

FIG. 16C shows energy densities at different laser power and scanning speed;

FIG. 17 shows microstructures of the printed stents; and

FIG. 18 shows the distribution of nickel wt % over 3 samples as printed and after heat treatment.

### DETAILED DESCRIPTION

The inventors envisioned that the stents of the present invention can be deployed in many areas within the human body. However, as a starting point, the femoropopliteal/femoral artery (FPA) is chosen for investigations for two main reasons. The FPA is a large artery located in the thigh provides majority of the arterial blood supply to the lower part of the extremity. First, the FPA undergoes one of the most extensive mechanical deformation in the human body during limb flexion, with twisting, bending and compression. The FPA experiences 2-4°/cm twist, 4-13% axial compression and has 22-72 mm bending radius during limb flexion. Measurements using intra-arterial markers showed even more severe deformation that were 2 to 7 times larger. Therefore, if the presently disclosed stent are made to handle the harsh conditions within the FPA, it is expected the stents are also suitable for use in other parts of the body. Second, as one of the largest arteries in the human body, the average common FPA has a diameter of 6.6 mm (3.9-8.9 mm), whereas the superficial FPA and deep FPA have average vessel diameters of 5.2 mm (2.5-9.6 mm) and 4.9 mm (2.7-7.6 mm) respectively. Current stents used in the FPA generally have diameters between 5 mm and 8 mm and are usually oversized compared to the diameter of the artery. Thus, stents with a range of diameters can be designed to ascertain its feasibility before scaling down for considerations in other parts of the body.

The material used to fabricate stents of the present invention is Nitinol. Nitinol is a metal alloy of nickel and titanium, where the two elements are present in roughly equal atomic percentages. Different alloys are named according to the weight percentage of Nickel, e.g. Nitinol 55 and Nitinol 60. The nickel content can be from about 40% to about 65%, and the titanium content can be correspondingly be from about 35% to about 60%. As used herein, Nitinol is used as a powder. The powder can have a particle size from about 10 μm to about 60 μm. The powder shows phase transformation peaks, i.e., the martensite start (Ms) and austenite start (As) temperatures at −17.9° C. and −5.6° C., respectively.

Nitinol alloys exhibit two closely related and unique properties: the shape memory effect and superelasticity (also called pseudoelasticity) at different temperatures. Shape memory is the ability of nitinol to undergo deformation at one temperature (generally a lower temperature), stay in its deformed shape when the external force is removed, then recover its original, undeformed shape upon heating above its “transformation temperature”. This is due to the reversion of martensite to austenite by heating, causing the original austenitic structure to be restored or reversed regardless of whether the martensite phase was deformed. The name “shape memory” refers to the fact that the shape of the high temperature austenite phase is “remembered” even though the alloy is severely deformed at a lower temperature.

Superelasticity is the ability for the metal to undergo large deformations and immediately return to its undeformed shape upon removal of the external load. Superelastic properties are generally observed when the temperature is above austenite temperature. Nitinol can deform 10-30 times as much as ordinary metals and return to its original shape. At high temperatures, nitinol assumes an interpenetrating simple cubic structure (austenite). At low temperatures, nitinol spontaneously transforms to a more complicated monoclinic crystal structure (martensite). There are four transition temperatures associated to the austenite-to-martensite and martensite-to-austenite transformations. Starting from full austenite, martensite begins to form as the alloy is cooled to the martensite start temperature (Mₛ), and the temperature at which the transformation is complete is the martensite finish temperature (M_{f}). When the alloy is fully martensite and is subjected to heating, austenite starts to form at the austenite start temperature (Aₛ), and finishes at the austenite finish temperature (A_{f}). This is commonly shown in a cooling/heating cycle as a thermal hysteresis. The hysteresis width depends on the precise nitinol composition and processing. Its typical value is a temperature range spanning about 20-50 K (20-50° C.).

In designing and/or fabricating the stent of the present invention, a self-expanding deployment method was considered. The stent will undergo shape setting in the expanded form before being crimped at/under room temperature for insertion into the catheter for deployment. The mechanical hysteresis behaviour of nitinol results in ‘biased stiffness’, whereby a stent that recovers from the crimped position or state will be much more resistant to compression than to expansion, which is useful for reducing chronic outward force (chronic outward force correlates to a measure of the radial force the stent projects outwards in its deployed configuration).

In contrast, balloon-expandable stents which are usually made from stainless steel or cobalt-chromium has the disadvantage that when the stent is expanded, it is plastically deformed and retains a permanent geometry. There is also a perceived risk for balloon-expandable stents in arteries to be permanently deformed through outside pressure resulting in a partially or completely block vessel, once the buckling strength of the stent is exceeded.

In the stent market currently, there is no industry standard on the specifications of a stent and the properties it should have. Stents manufacturers in the United States of America (US) would have to gain approval from the Food and Drug Administration (FDA) before they can release their products commercially. The FDA has provides a guidance regarding its current thinking on non-clinical engineering test that are submitted in investigational device exemption applications and premarket approval applications to support the safety and effectiveness of intravascular stents and their associated delivery systems. The comprehensive non-binding guidance includes an array of tests such as mechanical properties and stress/strain analysis, and the manufacturer has to provide details such as the test method, accept/reject criteria, sample size and results.

Therefore, the lack of an industry standard due to differing testing results and testing conditions of different manufacturers makes it difficult to verify if the designed stent is up to par or comparable with commercially available stents in terms of mechanical properties.

Accordingly, to verify the stent's mechanical properties, comparisons with commercially available stents under different mechanical loading conditions were made. Common testing modes include radial compression, crush resistance, bending, axial tension and compression, as well as torsion.

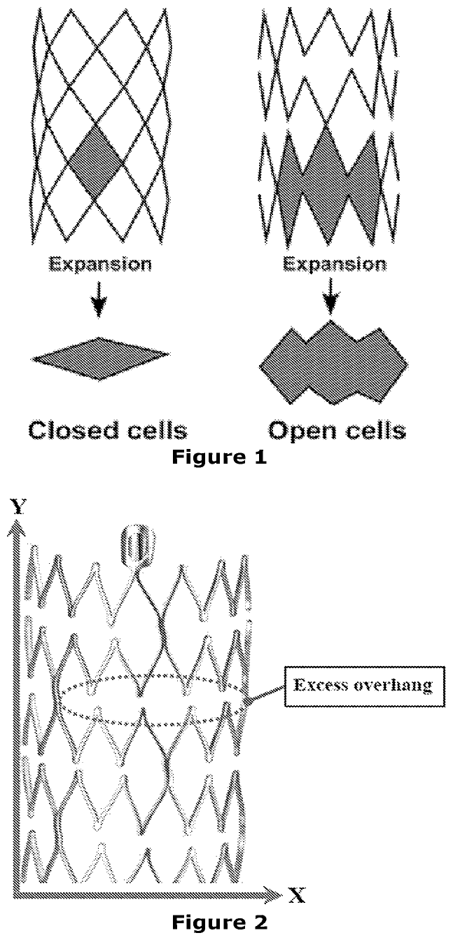

Stents can be classified with an open-cell or closed-cell design, which is dependent on the density of the struts (FIG. 1). Open-cell stents are characterized by large uncovered gaps whereas closed-cell stents have smaller free cell areas between the struts. Stent designs affect the flexibility and scaffolding of the stent, where closed-cell stents are less flexible and may develop kinks and incomplete expansion, while open-cell stents are flexible and conform to angulated vessels the best but may not provide sufficient scaffolding. However, the inventors believed that analysis of stents based on a single variable such as open-cell versus closed-cell, or one-dimensional attributes such as wall thickness or cell size will not give an accurate result. It is possible to design a closed-cell stent with a cell size larger than an open-cell stent, and a braided-wire stent and nitinol stent might both be classified as closed-cell but they share no meaningful design attributes. The inventors postulate that stents should be designed keeping in mind that the outcomes are not driven by single variables but instead an interrelated system of variables.

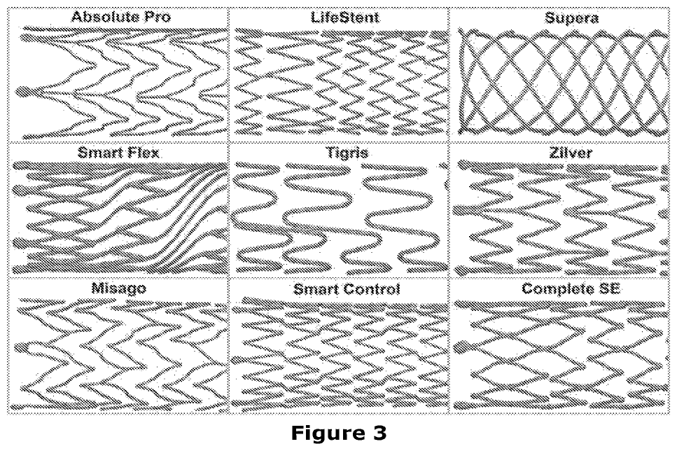

The inventors are of the opinion that the limitations of the stent fabricating method should also be considered. When using AM or SLM, the inventors have found that open-cell stents are not feasible for 3D printing due to the large overhang surfaces when printing from in any orientation. For example, when printing in orientation Y, there are excessive overhang areas which are not supported by the layer below, and which would result in a print failure (circled region in FIG. 2 is a possible print failure site). Whereas when printing in orientation X, there are excessive overhang areas as well, with the addition of curved cross sections in the horizontal plane that would require supports and would affect the print quality. To this end, the inventors have derived a stent design that is suitable for fabrication using 3D printing (such as AM or SLM) since the total area or overhang surfaces can be significantly reduced, which also maximizes flexibility.

To improve the flexibility of the closed-cell stent, it was postulated that the free cell area between each cell (in a closed cell stent) can be maximized to mimic an open-cell design, with bending features incorporated into the cell design. Not only will this provide the scaffolding required, it also promotes flexibility in this hybrid stent design. Additionally, since the strut thickness (wire diameter) and geometry can play an important role in the hemodynamic properties, the strut thickness can be minimised and the strut geometry can be optimized.

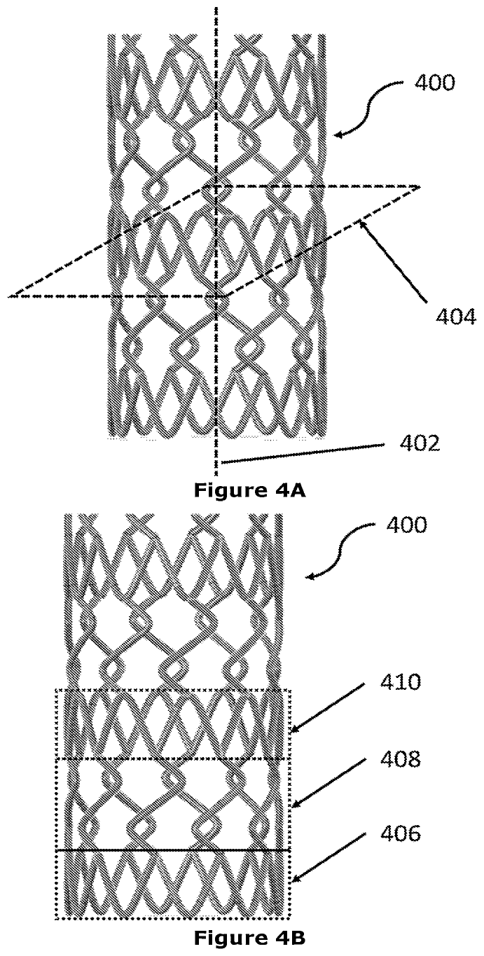

To investigate features that allowed closed-cell stents to achieve flexibility which measures up to that of an open-cell stent, features of commercial stents were studied. FIG. 3 compares nine stents available in the commercial market. Comparing open-cell designs, Tigris, Misago Absolute Pro and LifeStent have much lower bending stiffness than Smart Control and Smart Flex. From the comparison, it was found that stents with larger free cell areas have more flexibility.

The inventors have found that the flex sections should be designed such that the overhang areas are reasonable for 3D printing. The flex sections in commercial stents cannot be adopted for 3D printing as it has excessive overhang areas and small features that are spaced very closed together.

In an aspect, the present invention provides a 3D printed stent having a longitudinal dimension and a radial plane, the stent movable from a collapsed state to an expanded state, the stent comprising:
a) at least two circumferential sections that are radially expandable in order for the stent to move from the collapsed state to the expanded state; and
b) one or more flex sections, each flex section extending between two adjacent circumferential sections, each flex section being longitudinally expandable in order for the stent to move from the collapsed state to the expanded state;
wherein the flex section comprises a plurality of circumferentially arranged flex units, each flex unit comprising at least three bends; and
wherein when in the expanded state, the at least three bends each independently has an angle relative to the radial plane of about 15° to about 90°.

In some embodiments, the 3D printed stent comprising:
a) at least two circumferential sections that are radially expandable in order for the stent to move from a collapsed state to an expanded state; and
b) one or more flex sections, each flex section extending between two adjacent circumferential sections, each flex section being longitudinally expandable in order for the stent to move from the collapsed state to the expanded state;
wherein each flex section comprises a plurality of circumferentially arranged flex units, each flex unit comprising a wire having a wave-like structure; and
wherein in the expanded state, the flex unit forms an angle of about 15° to about 90° relative to a local radial plane at a junction with each of the adjacent circumferential sections.

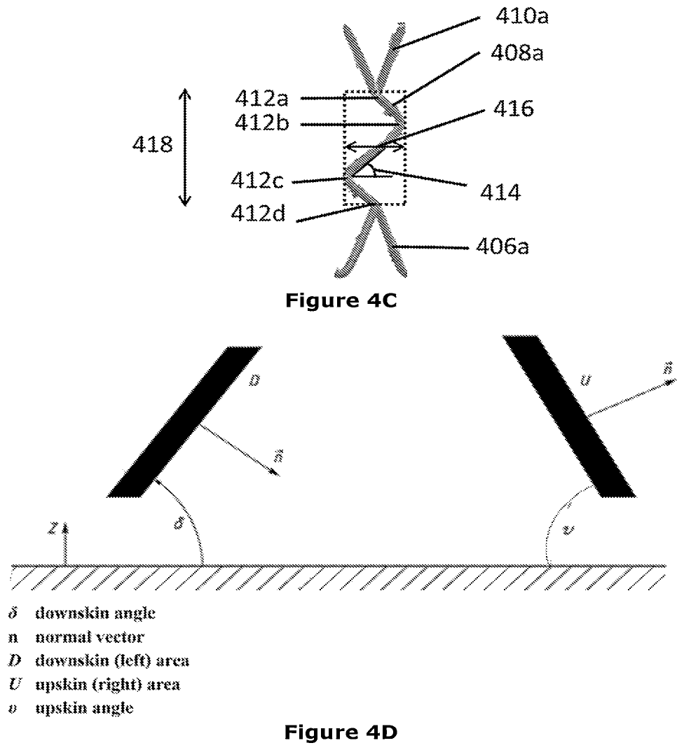

FIG. 4A illustrates an exemplary stent 400 of the present invention. The stent 400 has a longitudinal dimension 402 and a radial plane 404. The radial plane 404 is transverse to and perpendicular to the longitudinal dimension 402. The stent 400 can be 3D printed, layer-by-layer, along the longitudinal dimension 402. The stent 400 is shown in its expanded state. The stent 400 can be 3D printed in its expanded state, and subsequently collapsed to its collapsed state for insertion in a human body. For the stent to function as intended, the inserted stent in the collapsed state is then expanded, thus providing support to a blood vessel. In this regard, the stent is movable between a collapsed state and an expanded state, or at least movable from a collapsed state to an expanded state.

FIG. 4B shows that the expanded stent 400 comprises at least two circumferential sections 406 and 410. The circumferential sections 406 and 410 are movable radially in the radial plane for transiting between the collapsed state and the expanded state. To this end, the circumferential sections 406 and 410 are expandable radially. The circumferential sections 406 and 410 may also be movable in the longitudinal dimension 402 when transiting between the collapsed state and the expanded state. To this end, the circumferential sections 406 and 410 are contracting longitudinally when expanded.

The circumferential sections 406 and 410 are connected to each other by a flex section 408. Accordingly, flex section 408 is sandwiched between or disposed between circumferential sections 406 and 410. The flex section 408 extends between the two adjacent circumferential sections 406 and 410. The flex section 408 is movable along the longitudinal dimension 402 when transiting between the collapsed state and the expanded state. In this sense, the flex section is longitudinally expandable in order for the stent to move from the collapsed state to the expanded state.

It should be noted that the stent in moving from a collapsed state to an expanded state has an overall increase in length in the longitudinal dimension. Thus, if the circumferential section is contracting longitudinally, an increase in the longitudinal length of the flex section is greater than the decrease in the longitudinal length of the circumferential section.

As is shown in FIGS. 4A-B and 6A-B, the flex section can be formed with rounded corners or features. Traditionally, stents are formed with jiggered or see-saw edges due to limitations in the manufacturing process. Using 3D printing methods allows for a more rounded edge when printing the stent. This advantageously prevents tearing of the blood vessels when in use, which can be caused by the sharp edges of traditional stents.

The flex section 408 comprises a plurality of flex units. The flex units are circumferentially arranged, with its length 418 parallel to the longitudinal axis of the stent. FIG. 4C illustrates a single flex unit 408a within the flex section 408. The flex unit 408a can be formed from a wire having a wave-like structure. FIG. 4C shows an example in which the flex unit is wire with a sinusoidal wave-like structure. The flex unit is connected to the circumferential sections at both of its first and second ends, and forms an angle of about 15° to about 90° relative to a local radial plane at a junction with each of the adjacent circumferential sections. The local radial plane of the stent is thus formed with the point of intersection making up the angle within the radial plane.

At this angle, the flex units are 3D printable without experiencing overhang issues. The flex unit 408a is able to expand or contract in the longitudinal direction of 418 for expanding or contracting the stent along a longitudinal axis. The flex unit 408a is also able to expand or contract in the transverse direction of 416. It is not expected that this expansion and/or contraction in the direction of 416 will change the width of the stent.

The flex unit can alternatively be characterised as having at least three bends, for example with at least two of the bends in contact with the circumferential sections. In this example, the flex unit has 4 bends 412a, 412b, 412c and 412d. In this expanded state, the bends (412a, 412b, 412c and 412d) can each independently have an angle (downskin angle) 414 relative to the radial plane 404 of about 15° to about 90°.

The “downskin angle” is illustrated as δ in FIG. 4D. The downskin angle is the angle which a downward facing side of an element makes with a horizontal surface. This definition is based on the standard ISO/ASTM 52911-1:2019.

The stent is fabricated from Nitinol, a metal alloy of nickel and titanium.

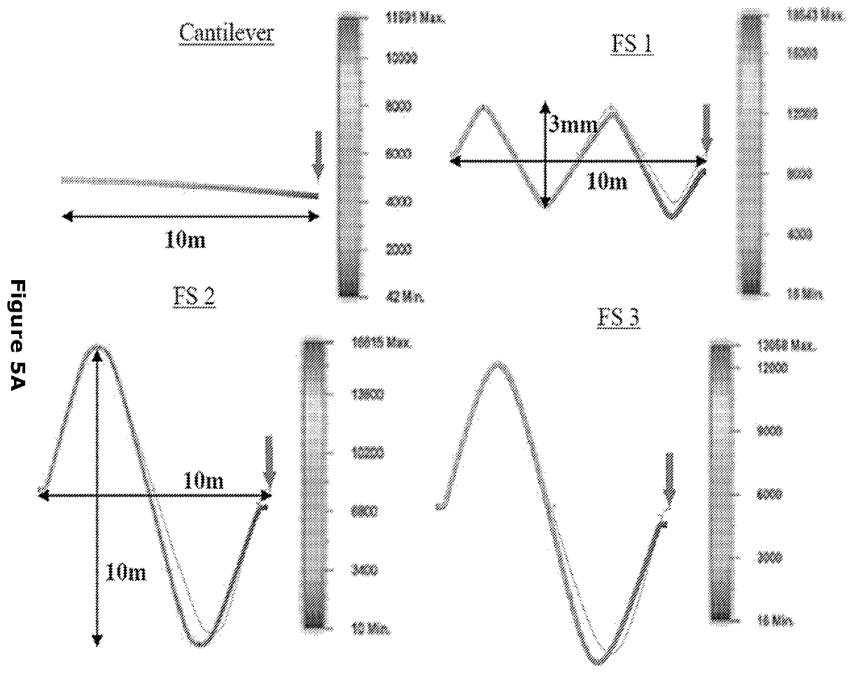

To determine the flexibility and suitability of flex section for 3D printing, 7 different flex units were fabricated for comparison. FIG. 5A illustrate the stress concentration of each model, while FIG. 5B illustrate the amount of displacement. The deformation of each FS could also be observed from the simulations, where the faint black line denotes the original position of the FS.

Simulation results of 7 different flex units are shown in Table 1. When a greater amplitude is provided in the model (corresponding to the transverse breadth of a flex unit as shown in for example 416) or downskin angle of the flex segment (FS) is increased, a greater maximum displacement (which translates to a longitudinal expansion) without significantly changes to the maximum stress experienced by the part is observed. The largest displacement of the FS occurs at the point where the force was applied. This translates to a maximum displacement in the longitudinal dimension of the stent. Another observation is that sharp peaks (or smaller downskin angle) in the FS reduces the maximum stress incurred without substantial reduction in the maximum stress, and this observation is more distinct in FS with greater height or downskin angle. For example, and referring to 412b and 412c, if the curvature at these bends is made sharper or smaller, the maximum stress in the flex unit can be reduced.

TABLE 1
Comparison between flex units (flex
segments) and cantilever beam

| | Maximum | Maximum | Maximum |
|---|---|---|---|
| | Stress | Displacement | Overhang |
| | (MPa) | (mm) | (degrees) |
| Cantilever Beam | 11691 (100%) | 18.16 (100%) | 0 |
| Flex Segment 1 | 18543 (159%) | 27.91 (154%) | ~57 |
| Flex Segment 2 | 16615 (142%) | 41.67 (229%) | ~71 |
| Flex Segment 3 | 13058 (112%) | 41.21 (226%) | ~71 |
| Flex Segment 4 | 16785 (144%) | 26.41 (145%) | ~53 |
| Flex Segment 5 | 14859 (127%) | 25.09 (138%) | ~54 |
| Flex Segment 6 | 16362 (140%) | 23.98 (132%) | ~46 |
| Flex Segment 7 | 15902 (136%) | 23.7 (131%) | ~47 |

Therefore, flex units with sharp peaks (or smaller downskin angle) would be preferred since they produces better results by offering flexibility without incurring as much stress. Flexibility of the stent can also be altered by varying the height (breadth in the transverse plane) and/or downskin angle of the flex units, and the variation has to be within the limitations of the 3D printer since excessive overhang might cause the print to fail. Hence, incorporation of flex units of similar designs that are within the 3D printing boundaries have great potential of improving the flexibility of the initial stent design.

In some embodiments, the downskin (or upskin) angle is about 15° to about 80°, about 15° to about 70°, about 15° to about 60°, about 15° to about 50°, about 15° to about 45°, about 15° to about 40°, about 15° to about 35°, about 15° to about 30°, or about 15° to about 25°. In other embodiments, a sharp peak (or smaller downskin angle) as discussed herein refers to an angle of about 15° to about 30°. While a small downskin angle is particularly advantageous, a downskin angle of about 30° to about 60° can also be favourable for use in blood vessels with less demanding conditions.

In some embodiments, the flex section comprises a plurality of flex units. In other embodiments, each flex section comprises 5 to 12 flex units. In other embodiments, each flex section comprises 5 to 11 flex units, 5 to 10 flex units, 6 to 10 flex units, 7 to flex units or 8 to 10 flex units. In other embodiments, the flex section comprises 6 to 12 flex units, 6 to 11 flex units, 7 to 12 flex units, 8 to 12 flex units, 9 to 12 flex units, or 10 to 12 flex units.

In some embodiments, the wave-like structure is a sinusoidal wave-like structure or a helical wave-like structure.

In some embodiments, each flex unit has a wavenumber of about 0.5 unit to about 2 units. The wavenumber (also wave number or repetency) is the spatial frequency of a wave. FIG. 4C shows a flex unit with a wavenumber of 1 unit.

An angle is also present within the wave-like structure of the flex unit. When the flex unit has a wavenumber of at least 0.5 unit, a peak is present. The peak can be characterised by an angle of about 15° to about 90° relative to a local radial plane at the peak. The local radial plane of the stent is thus formed with the highest point of the peak within the radial plane.

The flex units can each independently have a periodic structure. In this case, the flex unit can have 4 bends 412a, 412b, 412c, 412d oriented along the longitudinal dimension as shown in FIG. 4C. For example, the periodic structure can be a periodic wave structure, such as a sinusoidal structure. The wave pattern can also be in the form of a spiral, helical or double helical pattern.

The flex units can each independently have a period of about 0.5 to about 2. The flex units can have a period of about 0.5, about 1, about 1.5, or about 2.

Each flex unit can comprise at least three bends. In other embodiments, each flex unit can independently comprise 3 or 4 or 5 or 6 bends. The bends in the flex units can also be considered as peaks and troughs (dependent on their orientation). The peak and trough can refer to the highest and lowest point of the periodic structure. When the flex unit comprises 4 bends, it can be considered to substantially be of a single sine wave.

When in the expanded state, the flex unit can have a transverse breadth 416 of about 2 mm to about 15 mm. The transverse breadth 416 is measured as the maximum distance of the flex unit perpendicular to the longitudinal axis of the stent. As shown in FIG. 4C, when the flex unit has a wavenumber of 1 unit or more, the transverse breadth can be the peak to peak amplitude. The breadth 416 can also be defined as the distance or displacement between a highest point and a lowest point of the bend structures of the flex unit (or also the height or amplitude). In other embodiments, the breadth is about 3 mm to about 15 mm, about 3 mm to about 14 mm, about 3 mm to about 13 mm, about 3 mm to about 12 mm, about 4 mm to about 12 mm, about 5 mm to about 12 mm, about 6 mm to about 12 mm, about 7 mm to about 12 mm, about 8 mm to about 12 mm, or about 9 mm to about 12 mm.

When in the expanded state, the flex section and/or the flex units can have a length along the longitudinal axis of the stent of about 1 mm to about 15 mm, about 2 mm to about 15 mm, about 4 mm to about 15 mm, about 5 mm to about 15 mm, about 7 mm to about 15 mm, or about 10 mm to about 15 mm. The longitudinal length 418 is measured as the distance of the flex unit parallel to the longitudinal axis of the stent. As shown in FIG. 4C, when the flex unit has a wavenumber of 1 unit, the length is the wavelength. The length 418 is also defined as the inter-circumferential section distance (of the flex section between the adjacent circumferential sections); i.e. the distance between two adjacent circumferential sections and which is occupied by the flex section.

Stents with varying flex section and circumferential sections were studied to understand how these components affect stent performance. The following factors affecting stent performance were studied:
1) Number of circumferential units (in the circumferential section); i.e. width of stent
2) Width (longitudinal length 418) and height (transverse breadth 416) of flex unit
3) thickness of flex unit (cross sectional diameter of wire)

As shown in Table 2, different stent designs were modelled with varying features. For consistency in comparison, all designs have a stent diameter of 8 mm with 8 circumferential sections (or strut segments) and 7 flex sections (or flex segments). Designs 1-3 are used to compare how the cross section diameter of the flex unit (strut thickness) affects the properties of a stent, designs 1 and 4 for comparison of how the overhang angle affects the stent properties. Designs 5-7 allows a comparison between stent properties and the number of circumferential units.

TABLE 2
Stent examples

| Design | 1 | 2 | 3 | 4 | 5 | 6 | 7 |
|---|---|---|---|---|---|---|---|
| Pattern | A | A | A | A | B | B | B |
| Overhang angle | ~40° | ~40° | ~40° | ~35° | | | |
| Circumferential units | 10 | 10 | 10 | 10 | 10 | 8 | 6 |
| Strut thickness | 0.3 mm | 0.25 mm | 0.15 mm | 0.3 mm | 0.3 mm | 0.3 mm | 0.3 mm |

Designs 1-5 and designs 5-7 have different patterns, which are patterns A and B respectively. Pattern A is illustrated in FIG. 4A, and features alternating cell designs for each subsequent unit along the stent's print direction (longitudinal dimension).

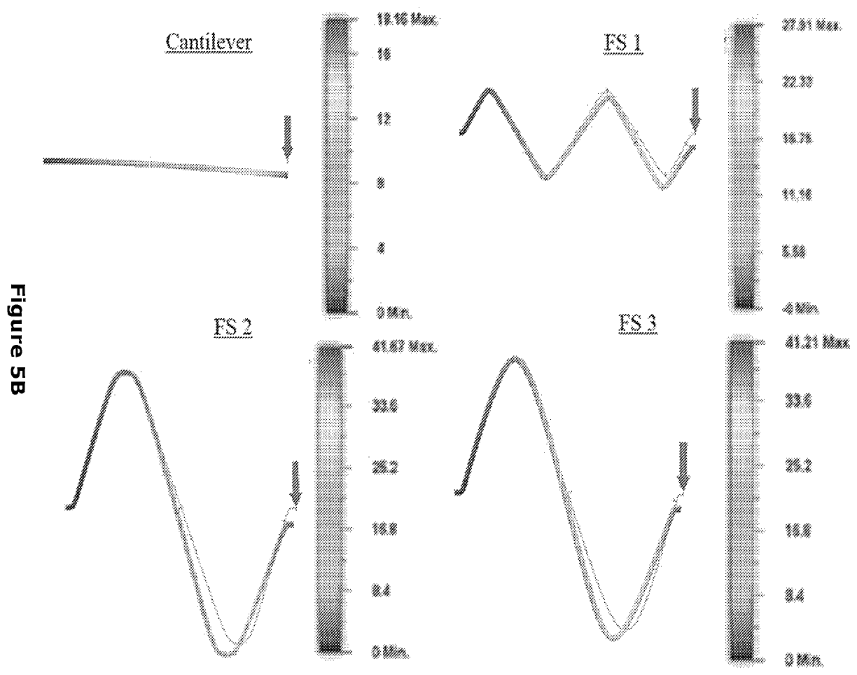

Pattern B is illustrated in FIG. 6A, and features same cell designs for all units along the print direction, and its flex sections additionally having allowance at both ends. A comparison of Pattern A and B thus allows for a study into the feasibility of 3D printing small spaces between flex sections and circumferential sections, as well as how a non-symmetrical pattern affects the performance of the stents.

In particular, FIG. 6A shows another embodiment of a stent 600. The stent 600 has a first circumferential section 602 and a second circumferential section 606. The first and second circumferential sections 602 and 606 are spaced apart, separated by a flex section 604. Flex section 604 is disposed or sandwiched between the first and second circumferential sections 602 and 606 and connects the two circumferential sections 602 and 606.

A flex unit 604a is illustrated in FIG. 6B. The flex unit 604a extends between a first circumferential unit 602a and a second circumferential unit 606a. Each flex unit comprising a wire having a wave-like structure. In this expanded state, each flex unit forms an angle of about 15° to about 90° relative to a local radial plane at a junction with each of the adjacent circumferential sections. The flex unit 604a can further comprise a first end and a second end. The first end of the flex unit 604a can comprise a first extension 612 connected to one of the two adjacent circumferential sections (or as shown, to a circumferential unit 606a). The second end can comprise a second extension 614 connected to the other of the two adjacent circumferential sections (or as shown, to a circumferential unit 602a).

Alternatively, the flex unit 604a comprises 4 bends 608a-d. The bends (608a-d) can each independently have an angle (downskin angle) 610 relative to the radial plane of about 15° to about 90°.

In some embodiments, the first extension and the second extension each independently has a length of about 0.1 mm to about 5 mm. In other embodiments, the length is about 0.1 mm to about 4.5 mm, about 0.1 mm to about 4 mm, about 0.1 mm to about 3.5 mm, about 0.1 mm to about 3 mm, about 0.1 mm to about 2.5 mm, about 0.1 mm to about 2 mm, about 0.5 mm to about 2 mm, or about 1 mm to about 2 mm.

In some embodiments, the stent comprises a combination of Pattern A flex section and Pattern B flex section. In other embodiments, one flex section comprises a plurality of circumferentially arranged flex units with no extensions, while another flex section comprises a plurality of circumferentially arranged flex units with a first extension 612. In another example, one flex section comprises a plurality of circumferentially arranged flex units with no extensions, while another flex section comprises a plurality of circumferentially arranged flex units with a first extension 612 and a second extension 614. This advantageously provides greater flexibility in personalising a stent for a patient based on his condition.

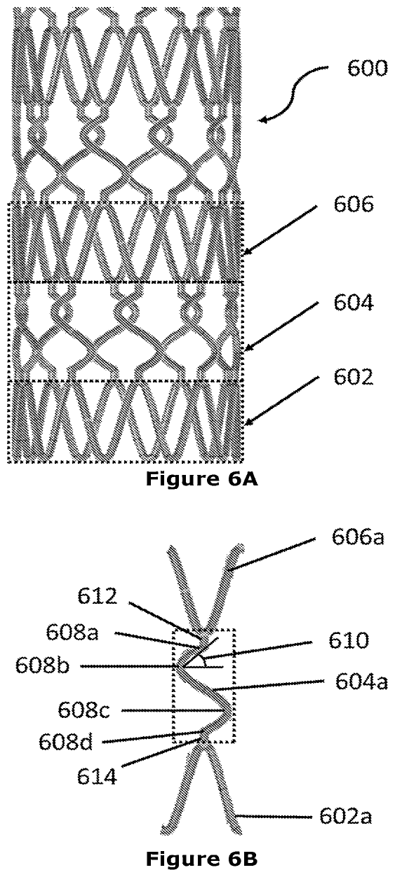

FIG. 7 shows another embodiment of the stent 700. Stent 700 comprises two circumferential sections 702 and 706. The circumferential sections 702 and 706 forms the terminal ends of the stent 700. Circumferential sections 702 and 706 are radially expandable in order for the stent to move from the collapsed state to the expanded state. A flex section 704 is extended between the two adjacent circumferential sections 702 and 706. Flex section 704 is longitudinally expandable in order for the stent to move from the collapsed state to the expanded state. The flex section 704 comprises a plurality of circumferentially arranged flex units. As shown in stent 700, each flex unit comprising six bends. In this expanded state, the bends each independently has an angle relative to the radial plane of about 15° to about 90°.

The flex units 704 can be connected to circumferential units 702 and 706 in a periodic structure. For example, the flex units in flex section 704 can be in a wave pattern, which can be in the form of a spiral, helical or double helical pattern. Each end of the flex unit in flex section 704 can be connected respectively to a circumferential unit in the circumferential sections 702 and 706. For example, the connection can be at a turning point (peak or trough) of the circumferential unit.

FIG. 7B illustrates another embodiment of the present invention. The terminal circumferential sections are connected to each other by a flex section. The flex section is extended between the two adjacent circumferential sections. The flex section comprise at least 2 flex units. Each end of the flex unit in flex section is connected respectively to a circumferential unit in the circumferential sections. For example, circumferential unit at A2 of one circumferential section can be connected to circumferential unit at B2 at another circumferential section. The circumferential units at A1, A3,131 and B3 are not connected. The 2 flex units can be arranged helically about the longitudinal dimension of the stent. As shown in FIG. 7B, the flex units are arranged in an anti-congruent arrangement such that one flex unit is a mirror image of the other flex unit. Alternatively, the flex units can be arranged in a congruent arrangement such that, for example, a double helix is formed.

The various examples as disclosed herein illustrates the flexibility of the stent design when it comprises circumferential sections and at least one flex section. Further by varying, for example, the number of bends, the transverse breadth of the flex units, the number of flex units, the number of circumferential units, the longitudinal length of the sections, various stents can be fabricated to suit the specific requirements of a patient.

In some embodiments, the plurality of flex units each independently has a wire with a cross sectional diameter of about 0.2 mm to about 0.4 mm. For example, the wires of the plurality of flex units can have a cross sectional diameter of about 0.2 mm to about 0.4 mm.

Based on simulation studies, the inventors have found that a wire with a thinner cross section diameter of the flex unit (strut thickness) result in a larger displacement of the model when placed under bending, compression and tension, which translates to greater flexibility. This allows the stent to be subjected to a bending and/or compression force easily without breaking.

Similarly, flexibility can also be imparted by having wires with thinner cross section diameter of the circumferential units. In this regard, the wires forming the circumferential units can have a thinner cross sectional diameter. Further, lesser circumferential units in the circumferential sections also results in greater flexibility when placed under bending, compression and tension conditions. No distinct correlations can be observed from the displacement of the models when placed under torsion. When placed under a torsional force, designs 5 to 7 have a more even distribution of areas with higher stress concentrations across the entire stent structure, whereas the areas of higher stress concentrations are concentrated at areas closer to where the torsional force is acting for designs 1 to 4. Similarly, designs 5 to 7 have stress concentrations that are better distributed across all the flex segments across the entire structure, which is probably attributed to their uniform and symmetrical designs. The designs that are uniform also have a more even deformation model as observed from the simulation models. Therefore, it appears that a uniform circumferential section (strut) design (as provided using a periodic design) can result in a more uniform stress distribution and thus more predictable and favourable results. Flexibility of the stent in terms of bending, compression and torsion can also be manipulated by varying the strut longitudinal thickness as well as the number of circumferential units.

In some embodiments, each of the at least two circumferential sections comprises a plurality of circumferential units. The circumferential units are circumferentially arranged within the circumferential sections. FIG. 4C shows circumferential units 406a and 410a. FIG. 6B shows circumferential units 602a and 606a.

In some embodiments, the at least two circumferential sections each independently comprises about 5 circumferential units to about 12 circumferential units. In other embodiments, the circumferential section comprises about 5 circumferential units to about 11 circumferential units, about 5 circumferential units to about 10 circumferential units, about 6 circumferential units to about 10 circumferential units, about 7 circumferential units to about 10 circumferential units, or about 8 circumferential units to about 10 circumferential units. The number of units will depend on the desired diameter of the expanded stent, and to this end, the circumferential units can be adjusted to fabricate stents of different diameters.

Each circumferential section can be made up of circumferential units having a wave-like structure. For example, the circumferential units can have a sinusoidal wave-like structure. The circumferential units can be characterised by a length which can be a multiple of a wavenumber, and a peak to peak amplitude. The length forms a closed loop and extends circumferentially relative to the longitudinal axis of the stent. The circumferential units can be spaced apart from each other at regular intervals.

When the circumferential units have a sinusoidal wave-like structure, each circumferential section can have a wavenumber of about 5 unit to about 12 units.

An angle is also present within the wave-like structure of the circumferential section. The peak can be characterised by an angle of about 15° to about 90° relative to a local transverse plane at the peak. The local transverse plane of the stent is thus formed with the highest point of the peak within the transverse plane.

In some embodiments, the circumferential units each independently has a wire having a cross sectional diameter of about 0.2 mm to about 0.4 mm. In other embodiments, the circumferential units have a cross sectional diameter of about 0.22 mm to about 0.4 mm, about 0.24 mm to about 0.4 mm, about 0.26 mm to about 0.4 mm, about 0.28 mm to about 0.4 mm, about 0.3 mm to about 0.4 mm, about 0.32 mm to about 0.4 mm, about 0.34 mm to about 0.4 mm, or about 0.36 mm to about 0.4 mm.

In some embodiments, the at least two circumferential sections each independently has a periodic structure. In other embodiments, the at least two circumferential sections each independently has a periodic wave structure.

As shown in FIGS. 4A-B and 6A-B, the circumferential sections can be formed with rounded edges or features. For example, the circumferential section can have a sinusoidal structure. To this end, the circumferential unit can comprise a peak (bend at a high point) and a trough (bend at a low point).

In some embodiments, the circumferential units in the at least two circumferential sections are arranged such that the circumferential units in one circumferential section are anti-phase relative to the circumferential units in the other circumferential section. It was found that such an arrangement can provide additional support and strength to the stent, and can further reduce torsional breakage. Alternatively, the circumferential units are arranged such that they are in phase with respect to each other, or arranged such that they are substantially out of phase with respect to each other.

Depending on the circumferential sections, the number of flex units in the flex section may vary. In some embodiments, when the at least two circumferential sections comprises periodic or wave-like structures and are anti-phase with respect to each other, the stent is formed such that one end of a flex unit connects to a peak in a circumferential section and another end of the flex unit connects to a trough in another circumferential section. In other embodiments, when the circumferential section comprises periodic or wave-like structures and are in phase with respect to each other, the stent is formed such that one end of a flex unit connects to a peak in a circumferential section and another end of the flex unit connects to a peak in another circumferential section. In other embodiments, when the circumferential section comprises periodic or wave-like structures and are in phase with each other, the stent is formed such that one end of a flex unit connects to a trough in a circumferential section and another end of the flex unit connects to a trough in another circumferential section.

In some embodiments, when the circumferential section comprises periodic or wave-like structures, the stent is formed such that each peak are connected to a flex unit. Alternatively, alternate peak can be connected to a flex unit, or a third of the peaks are connected to flex units. In other embodiments, when the circumferential section comprises periodic structures, the stent is formed such that each trough are connected to a flex unit. Alternatively, alternate trough can be connected to a flex unit, or a third of the troughs are connected to flex units. In other embodiments, with the exception of the terminal circumferential section, when the circumferential section comprises periodic structures, the stent is formed such that each peak and each trough are independently connected to a flex unit. In other embodiments of the stent, when adjacent circumferential sections comprise different circumferential units, a peak of a circumferential unit on a circumferential section is connected via two (or more) flex units to two (or more) peaks or troughs of two (or more) circumferential units on the adjacent circumferential section. To this end, one end of the stent is wider than the other end. This stent variation can advantageously provide support at, for example, an intersection between a vein (or artery) and a capillary.

In some embodiments, circumferential units in the at least two circumferential sections are alternatively connected to flex units in the flex section. In other embodiments, at least 20% of the circumferential units in the at least two circumferential sections are connected to flex units in the flex section, In other embodiments, at least 30%, at least 33%, at least 40%, at least 45%, at least 50%, at least 60%, at least 66%, at least 70%, at least 80%, or at least 90% of the circumferential units in the at least two circumferential sections are connected to flex units in the flex section.

The stent comprises at least two circumferential sections. In some embodiments, the stent comprises 2 to 10 circumferential sections. The stent can comprises 3 to 10 circumferential sections, 4 to 10 circumferential sections, 5 to 10 circumferential sections, 6 to 10 circumferential sections, or 7 to 10 circumferential sections. The stent can comprise 2, 3, 4, 5, 6, 7, 8, 9 or 10 circumferential sections. The number of circumferential sections depends on the desired length of the stent, which depends on its application in the body. As the presently disclosed stent is fabricated using an additive method, the number of circumferential sections can be tuned to suit its desired application.

In some embodiments, the at least two circumferential sections each independently has a length along the longitudinal axis of about 2 mm to about 15 mm. This is also referred to as the peak to peak amplitude when the circumferential section has a wave-like structure. In other embodiments, the length is about 2 mm to about 14 mm, about 2 mm to about 13 mm, about 2 mm to about 12 mm, about 2 mm to about 11 mm, about 2 mm to about 10 mm, about 2 mm to about 9 mm, about 2 mm to about 8 mm, about 2 mm to about 7 mm, about 2 mm to about 6 mm, or about 2 mm to about 5 mm.

The stent can be formed with wires of circumferential units of varying cross sectional diameters. For example, a cross sectional diameter of the circumferential units in a circumferential section at an end portion of the stent can be thinner than a cross sectional diameter of the circumferential units in a circumferential section at a middle portion of the stent. Advantageously, this allows for stent to collapse into a more compact state such that it is easier to insert into a blood vessel.

In some embodiments, the at least two circumferential sections are two terminal circumferential sections. In other embodiments, the at least two circumferential sections comprises two terminal circumferential sections. The terminal circumferential sections can have a different morphology compared to the non-terminal circumferential sections. The two terminal circumferential sections can have has a wave-like structure with peaks which are not connected to the flex units, wherein the non-connected peaks are around. Advantageously, this allows for a reduction of sharp edges so as to reduce damage to the blood vessel.

In some embodiments, the stent comprises 1 to 9 flex sections. The stent can comprises 2 to 9 flex sections, 3 to 9 flex sections, 4 to 9 flex sections, 5 to 9 flex sections, or 6 to 9 flex sections. The stent can comprise 2, 3, 4, 5, 6, 7, 8, or 9 flex sections. The number of flex sections depends on the desired length of the stent, which depends on its application in the body. As the presently disclosed stent is fabricated using an additive method, the number of flex sections can be tuned to suit its desired application.

In some embodiments, the flex sections each independently comprise a plurality of flex units. The number of flex units in a flex section can be different from the number of flex units in another flex section. For example, the flex sections can each have a different number of flex units. In this regard, a flex section can have 8 flex units while a neighbouring flex section can have 7 flex units.

In some embodiments, the circumferential sections each independently comprise a plurality of circumferential units. The number of circumferential units in a circumferential section can be different from the number of circumferential units in another circumferential section. For example, the circumferential sections can each have a different number of circumferential units. In this regard, a circumferential section can have 10 flex units while a neighbouring circumferential section can have 9 flex units.

In some embodiments, the cross sectional diameter of wires of the plurality of flex units at an end portion of the stent is smaller than a cross sectional diameter of wires of the plurality of flex units at a middle portion of the stent. Advantageously, this allows for stent to collapse into a more compact state such that it is easier to insert into a blood vessel.

Strut geometry plays an important role in determining blood recirculation zones and shear rates. The inventors have found that a non-streamlined strut deployed at the arterial surface in contact with flowing blood, regardless of the height of the strut, promotes creation of recirculation zones, low shear rates as well as prolonged particle residence time. Based on computational studies, a significant recirculation region is present both downstream and upstream of the non-streamlined rectangular geometry, and the regions increases with increasing height. Whereas for the streamlined circular arc geometry, recirculation zone is observed only for the arc with largest height while the other arcs do not demonstrate any flow separation. Taken altogether, a streamlined geometry with smaller slopes of cross section morphology (less than about 90°) will be more favourable for endothelialization which decreases the rate of both restenosis and LST.

Conventional stents have wires with a rectangular cross section. Additionally, one limitation of 3D printing is the resultant surface quality of the printed product. For 3D printed products such as stents where the hatch distance is the diameter of the wire for diameter of less than 0.2 mm, and for parts which require low surface roughness, post processing such as electropolishing (EP) is required. In most cases, the surface finish after EP will still not be able to meet the low roughness requirements if the particle adherence to the structure is severe. The inventors have found that the 3D printed stent can be further improved when the wires forming the stent have a partially curved cross sectional shape. Particularly advantageously, besides improving the blood flow, these cross sectional shapes (for example arc, elliptical or aerofoil) also reduce particles adherence to the structure (or less balling). This reduces the risk of tearing or rupturing of the blood vessel during insertion.

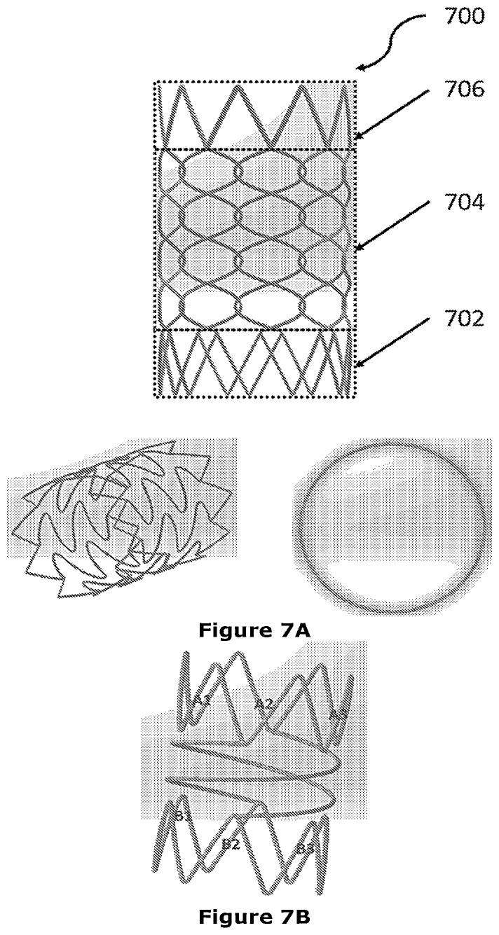

FIG. 8 shows other examples of cross sectional morphologies (geometries) that the wires forming the stent can take. In some embodiments, wires of the circumferential units and the flex units have a partially flat cross sectional shape. In other embodiments, the circumferential units and the flex units have a fully curved cross sectional shape. The curved cross sectional shape provides for a smaller slope as mentioned above, while gives the stent a streamlined geometry. In other embodiments, the circumferential units and the flex units have an elliptical, tear drop, aerofoil shape, partially flattened tear drop or circular cross section shape.

As the partially curved cross sectional shape is of an anisotropic shape, it can be characterised by a cross sectional thickness and a cross sectional width. In some embodiments, the cross sectional thickness is about 0.01 mm to 0.5 mm, about 0.01 mm to 0.4 mm, about 0.01 mm to 0.3 mm, about 0.01 mm to 0.2 mm, about 0.1 mm to 0.5 mm, or about 0.1 mm to 0.4 mm. In other embodiments, the cross sectional width is about 0.1 mm to 0.5 mm, about 0.1 mm to 0.4 mm, or about 0.2 mm to 0.4 mm.

The stent can have a diameter of about 4 mm to about 12 mm. In other embodiments, the diameter is about 5 mm to about 12 mm, about 6 mm to about 12 mm, about 7 mm to about 12 mm, about 8 mm to about 12 mm, or about 9 mm to about 12 mm.

The stent can have a length of about 7 mm to about 50 mm. In other embodiments, the length is about 7 mm to about 40 mm, about 7 mm to about 30 mm, or about 7 mm to about 20 mm. The length of stent can be extended depending on a patient-specific scale and requirement.

The stent can have a length to diameter aspect ratio of about 15:1 to about 30:1. In some embodiments, the aspect ratio is about 16:1 to about 30:1, about 17:1 to about about 18:1 to about 30:1, about 19:1 to about 30:1, about 20:1 to about 30:1, about 22:1 to about 30:1, about 24:1 to about 30:1, about 26:1 to about 30:1, or about 28:1 to about 30:1.

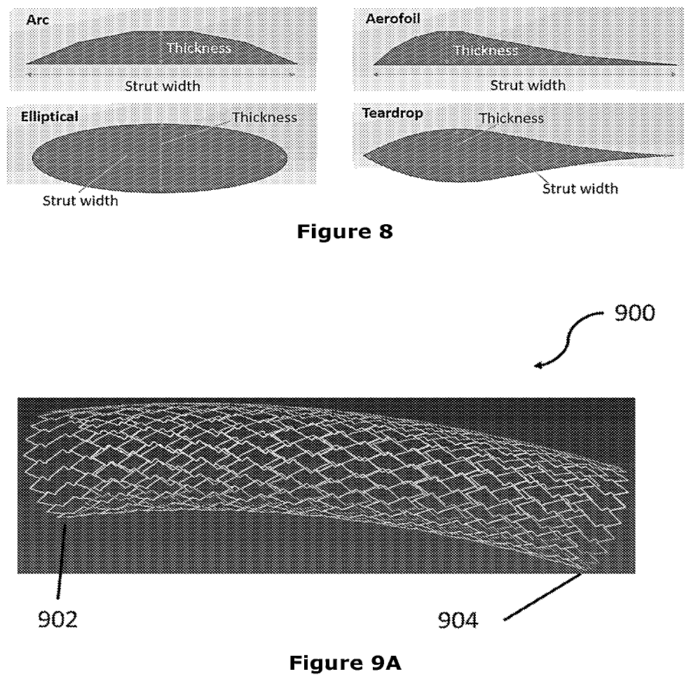

Current stents in the market are limited to straight stents. When a straight stent is placed in the curved blood vessel, a mechanical stress will be induced on the vessel wall. Overtime, this stress will cause injury to the vessel wall leading to restenosis. Curved stents with wires of less than 1 cm are seldom used due to the difficulties in fabricating and in delivering the curved stents to the site of repair in the vessel. It was found that by manipulation of the shape memory property as disclosed herein, the stent can have a straight profile when in a crimped state but adopt a curvature in an expanded state upon deployment in the vessel. In the crimped state, the straight profile facilitates the transfer of the stent in a catheter and positioning of the stent at the target vessel site. In the expanded state, the stent conforms to the curvature of the vessel thus alleviating stress on the vessel and on the stent. Accordingly, the stent, when in an expanded state, can have a curvature between its terminal ends (stent edges) and along its longitudinal dimension (FIGS. 9A and 9B). The stent 900 has a first end 902 and a second end 904. The stent 900 has a curvature along its longitudinal length, such that the second end 904 is offset from a longitudinal axis originating the first end 902. The radical and longitudinal curvatures can be varied depending on a patient vessel anatomy or geometry. Being able to customise a stent with a curvature is particularly advantageous as it reduces complications post-surgery. Human vascular environment and conditions may not be straight. At some of the lesions sites, stents may have to be bend to conform to the vessels. As such, a force will be exerted on the vascular tissue while the elastic stent tries to return to the original tubular straight-tube geometry. This is not ideal. Such non-straight stents fabricated according to patients vascular profile can allow for a reduce load on the vascular walls. This could potentially reduce mechanical stress exerted on the vessel, prolonging the integrity of the vessel during the service life of the stent. This helps to improve healing and reduce clinical complication such as vascular injury post-stenting.

The curved stent is able to be crimped (and thus straightened) for insertion into the catheter at room temperature and expanded to return to its original curved form at body temperature (when inside the vessel). This can be achieved without heat treatment post-fabrication of the stent by controlling the properties of the shape memory alloy.

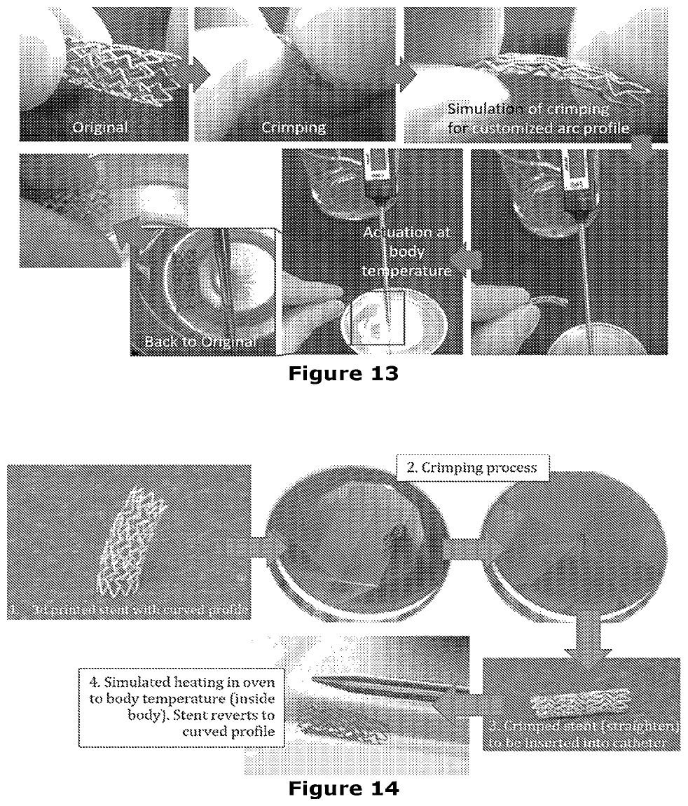

FIG. 14 shows an example of a curved 3D printed stent subjected to a crimping process. The stent can be fabricated with a curvature. After fabrication, the stent can be deformed or bent (without fracture) at room temperature (at 1). When crimped using a stent crimping machine (at 2), the stent reduces in size and straightens (at 3). Upon heating to at least body temperature, the crimped stent expands to its original diameter and curved profile (at 4).

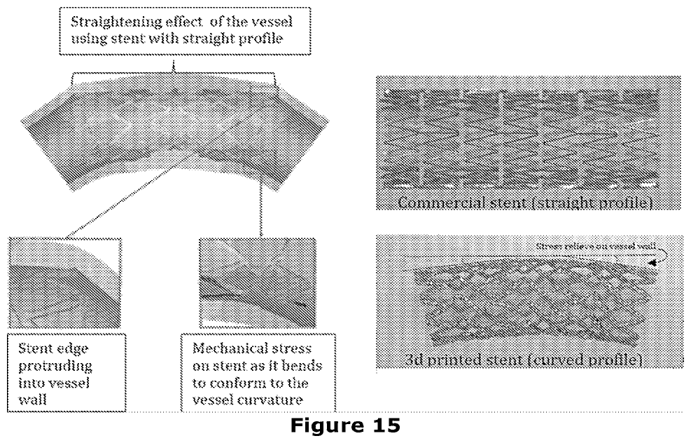

FIG. 15 shows (Left) simulation results of stent with straight profile deployed in curved vessel; and (Right) comparison of commercial and 3d printed stent with curved profile of the present invention. When a self-expanding stent with straight profile is inserted into the vessel at a location that is tortuous (with bends), the stent will have a tendency to straighten the vessel as the stent will always return to its original straight profile due to its superelasticity. This inevitably cause the stent edge to slightly protrude into the vessel wall. Overtime, due to stress on the vessel, the stent could damage the vessel lining, leading to restenosis. At the same time, the vessel will exert an opposite force to resist the stent from straightening resulting in the stent to bend until a neutral position between the vessel and the stent is reached. The bend leads to mechanical stress on the stent which may weaken the stent overtime. A curved stent could potentially reduce the mechanical stress on the vessel leading to a more favorable clinical outcome. The 3d printed curved stent can have both shape memory and superelastic properties at room temperature and body temperature respectively. At room temperature, the stent is capable to be deformed (crimped) without fracture. The crimping from curved to straight profile facilitates the insertion of the stent into a delivery device such as the catheter. Upon releasing the stent at the intended location in the vessel, the stent will be stimulated by the body temperature to return to its original diameter and curved profile to maintain the patency of the vessel. This reduces or eliminates stress on the vessel as well as on the stent. As the stent also has superelastic properties, the stent maintains it shape in the body even if it is subjected to compression forces.

To impart a curvature to a stent, the units forming a section can have different longitudinal lengths. In some embodiments, the lengths of respective flex units in a flex section are different from each other in the longitudinal dimension. For example, the length of a flex unit in a flex section can be different from its neighbouring flex unit. This can provide a curvature to the stent when expanded.

In some embodiments, the lengths of respective circumferential units in a circumferential section are different from each other in the longitudinal dimension. For example, the length of a circumferential unit in a circumferential section can be different from its neighbouring circumferential unit. This provides a curvature to the stent when expanded.

In some embodiments, the flex units in a flex section have a length in the longitudinal dimension which is different from the flex units in an adjacent flex section. In other embodiments, a flex section has a longitudinal length which is different from the longitudinal length of an adjacent flex section.

In some embodiments, the circumferential units in a circumferential section have a length in the longitudinal dimension which is different from the circumferential units in an adjacent circumferential section. In other embodiments, a circumferential section has a longitudinal length which is different from the longitudinal length of an adjacent circumferential section.

The transverse breadth of the flex units can also be varied to create suitable driven force, displacement and geometrical curvatures for stents. In some embodiments, the transverse breadth of respective flex units in a flex section are different from each other. In other embodiments, the transverse breadth of flex units in a flex section are different from those of flex units in an adjacent flex section. For example, the transverse breadth can be about 1 mm to about 7 mm, about 1 mm to about 6 mm, about 1 mm to about 5 mm, about 2 mm to about 5 mm, or about 3 mm to about 5 mm.

In some embodiments, the stent has a curvature between its terminal ends of about 1° to about 160°. In a stent with a single curve, the curvature refers to an angle between two convergent lines extending from an edge of the stent to an opposite edge of the stent. In a stent with multiple curves, the curvature can be measured between an edge to an inflexion point along the length of the stent or between two adjacent inflexion points. In other embodiments, the curvature is about 1° to about 150°, about 1° to about 140°, about 1° to about 130°, about 1° to about 120°, about 1° to about 110°, about 1° to about 100°, about 1° to about 90°, about 1° to about 80°, about 1° to about 70°, about 1° to about 60°, about 1° to about 50°, about 1° to about 45°, about 1° to about 40°, about 1° to about 35°, about 1° to about 30°, about 1° to about 25°, about 1° to about 20°, about 1° to about 15°, or about 1° to about °.

In some embodiments, the stent is characterised by a radius of curvature of about 1 mm to about 100 mm. In other embodiments, the radius of curvature is about 1 mm to about 90 mm, about 1 mm to about 80 mm, about 5 mm to about 80 mm, about 10 mm to about 80 mm, about 20 mm to about 80 mm, about 30 mm to about 80 mm, about 40 mm to about 80 mm, about 50 mm to about 80 mm, or about 60 mm to about 80 mm.

In some embodiments, the stent is characterised by a radius of curvature of about 1 cm to about 200 cm. In other embodiments, the radius of curvature is about 1 cm to about 180 cm, about 1 cm to about 160 cm, about 1 cm to about 150 cm, about 1 cm to about 140 cm, about 1 cm to about 130 cm, about 1 cm to about 120 cm, about 1 cm to about 110 cm, about 1 cm to about 100 cm, about 1 cm to about 90 cm, about 1 cm to about 80 cm, about 1 cm to about 70 cm, about 1 cm to about 60 cm, about 1 cm to about cm, about 1 cm to about 40 cm, about 1 cm to about 30 cm, about 1 cm to about 20 cm, or about 1 cm to about 10 cm.

As used herein, “radius of curvature” means the radius of a circle that touches a curve at a given point and has the same tangent and curvature at that point. The radius of curvature of a stent may refer to the radius of curvature of either side of the stent, or the radius of curvature of the longitudinal axis of the stent graft.

The stent can have a curvature at more than one location along its length. FIG. 9B shows another example of a stent with a double bend. The number of bends is not limited, and can be 3D printed accordingly based on the requirements of the vessel of a patient.

In some embodiments, the stent comprises an elemental composition of:
a) nickel of about 54 wt % to about 57 wt % of the composition; and
b) titanium of about 43 wt % to about 46 wt % of the composition.

In some embodiments, the stent comprises an elemental composition of:
a) nickel of about 55.2 wt % the composition; and
b) titanium of about 44.8 wt % of the composition.

The elemental composition of Nitinol stent was found to fall within medical grade Nitinol, as defined by ASTM F2063.

The present invention also provides a method of fabricating a 3d printed stent.

Accordingly, the present invention provides a method of 3D printing a stent, comprising:
a) providing a template of the stent; and
b) performing selective laser melting of a Nitinol powder based on the stent template in order to form the stent, the stent having a longitudinal dimension and a radial plane, the stent movable from a collapsed state to an expanded state;
wherein the stent template comprises:
i) at least two circumferential sections that are radially expandable in order for the stent to move from the collapsed state to the expanded state; and
ii) one or more flex sections, each flex section extending between two adjacent circumferential sections, each flex section being longitudinally expandable in order for the stent to move from the collapsed state to the expanded state;
wherein the flex section comprises a plurality of circumferentially arranged flex units, each flex unit comprising at least three bends; and
wherein when in the expanded state, the at least three bends each independently has an angle relative to the radial plane of about 15° to about 90°.

In some embodiments, the method of 3D printing a stent, comprises:
a) providing a template of the stent; and
b) performing selective laser melting of a Nitinol powder based on the stent template in order to form the stent, wherein the stent template comprises:
i) at least two circumferential sections that are radially expandable in order for the stent to move from a collapsed state to an expanded state; and
ii) one or more flex sections, each flex section extending between two adjacent circumferential sections, each flex section being longitudinally expandable in order for the stent to move from the collapsed state to the expanded state;
wherein each flex section comprises a plurality of circumferentially arranged flex units, each flex unit comprising a wire having a wave-like structure; and
wherein in the expanded state, the flex unit forms an angle of about 15° to about 90° relative to a local radial plane at a junction with each of the adjacent circumferential sections.

Advantageously, the method allows for greater flexibility in fabrication as process parameters can be used to focus on manipulating the Nitinol properties to achieve the desired outcome. Further advantageously, by 3D printing the stent, the stent can be customised to adapt to a patient's blood vessel conditions, by for example varying radical dimensions.

The inventors have found that to ensure the expanded stent has a definite cylindrical shape, it is particularly advantageous to fabricate the stent in its expanded form before shape-setting it through heat treatment.

In some embodiments, the stent is printed in its expanded state.

While the present invention is illustrated using SLM, other AM approaches such as laser engineered net shaping (LENS) E-beam melting (EBM), direct metal laser sintering (DMLS) and selective laser sintering (SLS) can also be used.

As mentioned, the stent is 3D printed from Nitinol, a shape memory alloy. Nitinol is an intermetallic compound with approximately equiatomic nickel and titanium, exhibits the unique properties of shape-memory and superelasticity which were originated from reversible phase change from an austenitic to a martensitic microstructure when subjected by temperature or external stress. At lower temperatures, nitinol have a readily deformable crystalline arrangement termed martensite. This structure can be achieved by cooling nitinol below the martensite start and finish temperatures, Mₛ and M_{f} respectively, which restructures the material into the low-temperature stable martensitic phase. By reheating the nitinol through its austenite start and finish temperatures, Aₛ and A_{f} respectively, the alloy passes through a characteristic transformation temperature range (TTR), causing the realignment of atomic planes that has occurred to be reversed. The crystal structure alters to a rigid and ordered cubic-like configuration known as austenite. This reversible process describes the shape-memory effect of nitinol.

Current stents in the market rely only on its superelastic properties for their deployment in a subject. The stent when subjected to large deformations are able to immediately return to its undeformed shape upon removal of the external load. It is this function that allows the stent to be compressed for insertion (via a delivery tube) and which subsequently ‘open’ when exiting the delivery tube for supporting a blood vessel. The shape memory effect of Nitinol is not used as there is an industry scalable method to control the A_{f} temperature within a suitable range.

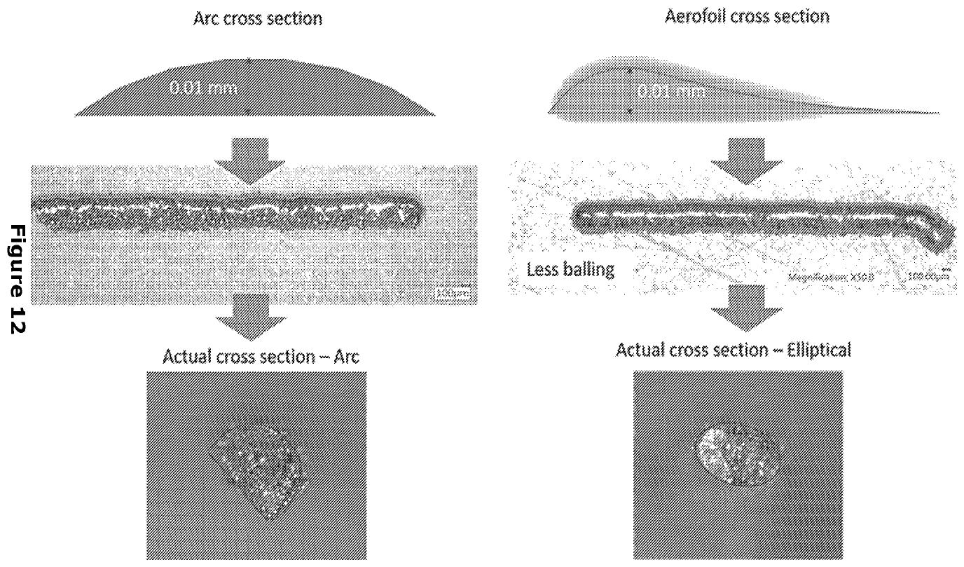

The inventors have found a method that allows deployment of stents of the present invention (via shape memory effect) to be achieved by heating the nitinol stent to a suitable temperature, such as a body temperature. To this end, the nitinol stent can be cooled and crimped (in the martensitic phase). The crimped stent maintains its collapsed state and can subsequently actuate or expand to its expanded state (austenite phase) when, for example, placed at body temperature (FIG. 13). The purpose of crimping of the stent (to be attached to a catheter) is to cater for the maneuverability and trackability in the lumen before deployment at the narrowed site in the vascular. This is applicable to 3D printed customised stent with inherent curvature (bent) based on patient's vascular profile.

In particular, the inventors have found that specific printing parameters can be used to vary the austenite and martensite temperatures. By doing so, the temperatures at which superelasticity takes effect can be controlled such that it is suitable for use in the human body. Additionally, by controlling the austenite and martensite temperatures, the shape memory properties of Nitinol can be advantageously utilised in a way to allow for easy insertion of a stent into a vessel.

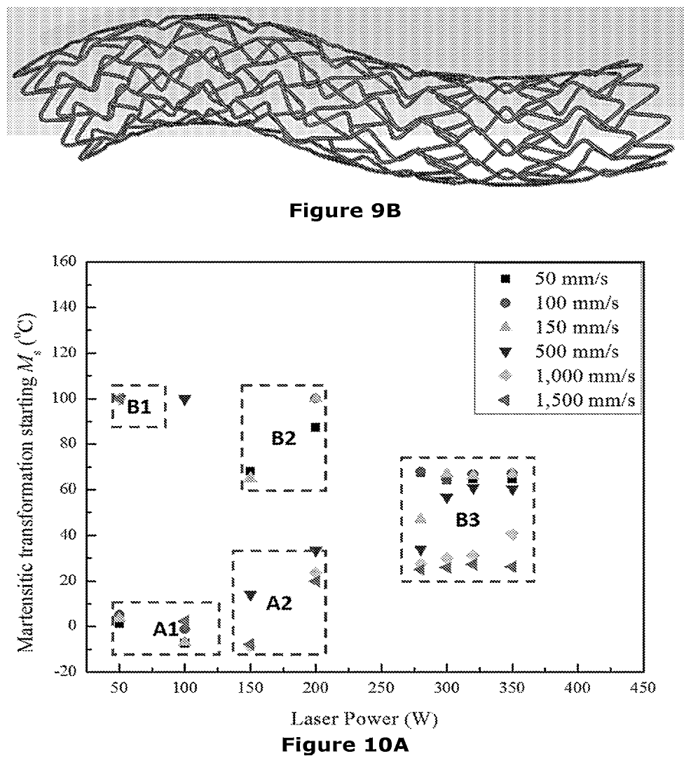

FIG. 10A-B shows embodiments of stents formed based on the present invention using different processing parameters. As will be discussed below, several factors can be modulated to 3D print stents from Nitinol. FIG. 10A illustrates that the resulting stents can have different groups of physical properties based on the disclosed methods.

Without wanting to be bound by theory, the inventors postulated that additive manufacturing processes can be used to fabricate stents. Additive manufacturing, known 3D printing, is defined as “a process of joining materials to make objects from 3D model data, usually layer upon layer, as opposed to subtractive manufacturing methodologies” (ASTM international). Powder bed fusion (PBF) method can be used to melt or fuse powders together in a layer-by-layer approach, and the printing process was named according to the energy types, i.e., E-beam melting (EBM), selective laser melting (SLM), direct metal laser sintering (DMLS) and selective laser sintering (SLS). SLM is a laser-powered powder bed fusion process that can produce highly dense metallic parts with delicate geometrical features. Metallurgical bonding of originally loose powders can be achieved by laser melting followed by rapid solidification. This invention has introduced the optimized printing parameter regimes in a selective laser melting system, to achieve superelasticity, shape memory effect, and surface finishing using process parameters under different regimes.

To optimize the printing process parameters for stents, wires having aspect ratio exceed 20:1 comparing the height to the diameter, were printed at angles ranged from 90°, 60°, 45° and 30°, respectively. Functional material characterizations were conducted, wherein the porosity of wires were examined by observing the microstructurel features and high resolution X-ray computed tomography, the elemental distribution of nickel and titanium was analyzed using energy dispersive X-ray detector (EDX) attached to scanning electron microscope (SEM), the phase formed was characterized using differential scanning calorimetry (DSC), and the mechanical performances were characterized using mechanical tensile tests.

The volumetric energy density can be defined as:

Eᵥ=P/(V×h×t)

Where, Eᵥ is energy density (J/mm³), P is the laser power (W), v is the scan speed (mm/s), and h is scan spacing or hatch distance (mm), and t is layer thickness (mm).

Without wanting to be bound by theory, it was found that the energy level, which is commonly used in the art, is not sensitive enough to predict the printing quality. This is especially so for Nitinol, as the shape memory and superelasticity properties are highly sensitive to powder composition and printing parameters. For example, it was found that even if using the same energy level, samples show different mechanical properties when different combinations of parameters were applied. Additionally, it was found that keeping the term of (v×h×t) as constant while changing any of three individual parameters does not necessarily lead to fabrication of identical parts in terms of microstructure and mechanical responses. It is believed that to improve consistency of printing, the relationship between the printing energy levels, elemental composition, the transformation temperatures and mechanical performances must be understood. This is aided using microscope, DSC, EDX and mechanical analysis. As disclosed herein, the energy levels of printing nitinol wires were tested from 6.67 to 2333.33 J/mm³ by changing the laser power and scanning speed, with fixed hatch distance of 0.1 mm and layer thickness of 0.03 mm. It was found that with adjustment of parameters, two printing regimes can be obtained. To this end, by selectively changing the printing conditions, optimized combinations for printing stents of different mechanical properties can be fabricated for different applications.

Several factors can be modulated to 3D print stents from Nitinol. FIG. 10A-B shows an example in which parameters such as laser power and scanning speed are varied. The laser power can be varied from about 50 W to about 400 W. The scanning speed can be varied from about 50 mm/s to about 1500 mm/s. As shown in FIGS. 10A and B, stents with different physical properties can be 3D printed based on different combinations of these parameters.

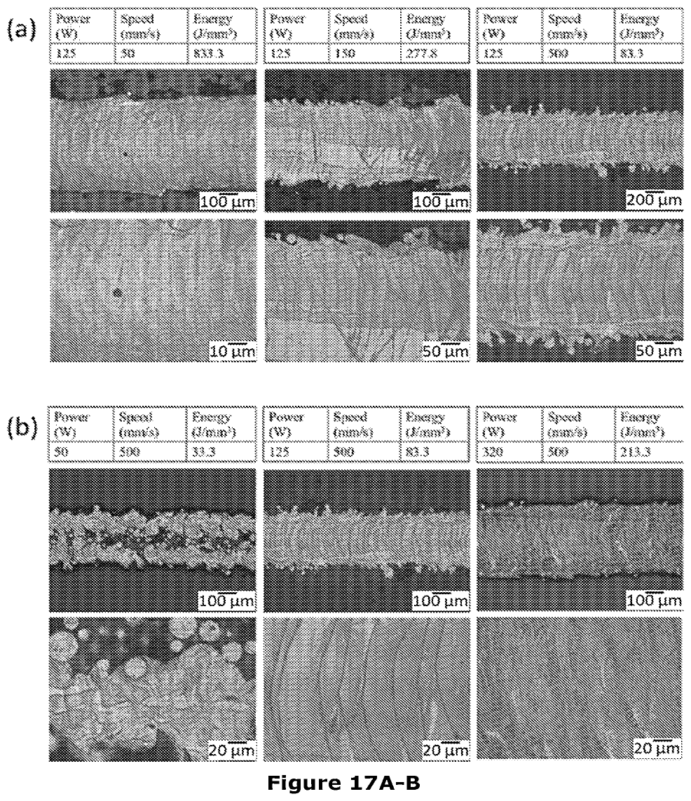

To elucidate desirable printing parameters, the microstructural features of printed samples at different parameters were compared to observe the microstructural evolution path with respect to the SLM process parameters. FIG. 17a shows the printed samples at the same laser power of 125 W, combined with a scanning speed of 50 mm/s, 150 mm/s, and 500 mm/s, corresponding to a decreased energy density from 833 J/mm³, 278 J/mm³, and 83 J/mm³, respectively.

The three samples all showed full density (no porosity in the structure) (FIG. 17a). The depth of layer boundary was at about 30 μm when printed at the speed of 500 mm/s, which was almost consistent with the layer thickness applied in the printing. With decreasing the speed to 150 mm/s, columnar grains were formed, suggesting that excess laser energy has re-melted the previous layers in the printing. When further reducing the scanning speed to 50 mm/s, the grain boundaries in the central region of the sample were merged. This over-heating may serve as the secondary heat treatment in the SLM process, therefore increasing the grain size. This additional heating has caused an increase in the phase transformation temperature, from −10° C. at P125 v150 and P125 v50 to 25° C. at P125 v500, respectively (P refers to the power (W) and v refers to speed (mm/s)).

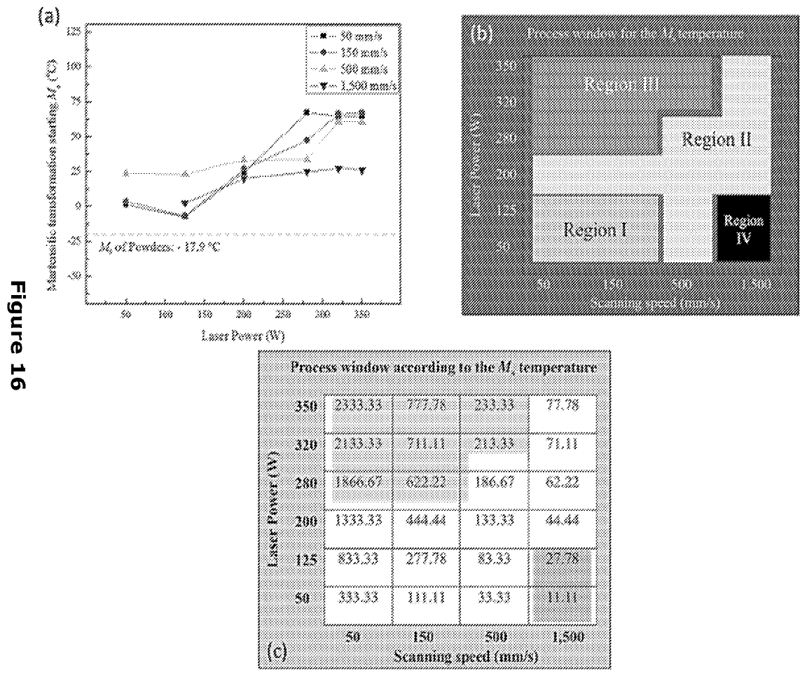

In contrast, FIG. 16b shows the samples printed at the same scanning speed of 500 mm/s but with laser power of 50 W, 125 W, and 320 W, which reveal the microstructural transition when printed using varied laser power. The microstructure of the printed samples was shown in FIG. 17b. At the scanning speed of 500 mm/s, high internal porosity was observed with printed at a laser power of 50 W, corresponding to a low energy density of 33.3 J/mm³. With increasing the laser power to 125 W (energy density at 83.3 J/mm³), a fully dense strut was printed with 30 μm layer thickness. Interestingly, the layer boundaries were fully merged when the laser power was increased to 320 W, although the energy density was at 213 J/mm³, which was still lower than 278 J/mm³ when printed at a laser power of 125 W and scanning speed of 150 mm/s. The same Mₛ temperature was obtained when printed at 50 W and 125 W at 500 mm/s scanning speed, suggesting that similar crystalline structures or phase has been formed at low laser power printing. This has further suggested that the microstructures were not significantly manipulated by the low laser power printing process. However, a significant jump in the Ms temperature was observed when the laser power was increased from 125 W to 320 W, which may originate from nickel evaporation when printed at high laser power.

Three struts were printed to examine the microstructures formed when printed at P100 v100, P200 v500, and P300 v1000, corresponding to the energy density of 333.3, 133.32, and 99.9 J/mm³. The Mₛ temperatures was at −1.24° C. for P100 v100, at 35.43° C. for P200 v500, and at 30.11° C. for P300 v1000. As shown in FIG. 17c, clear characteristic microstructural features were observed at the selected printing conditions, i.e., merging of layer boundaries at P100 v100; formation of columnar grains at P200 v500 and P300 v1000.

These three trends suggested melt pool boundary evolutions. Firstly, at low laser power printing, decreasing the scanning speed to 150 mm/s or below has caused inter-layer over melt, thus formed columnar grains. However, this is not likely to induce significant elemental redistribution in the strut. As a result, the phase transformation temperatures of struts processed under this regime were close to that of the powder. Secondly, high laser power (320 W) tended to re-melt the previous layers and likely caused redistribution of nickel elements in the grain boundaries. Moreover, nickel evaporation may be induced at high laser power. This facilitated secondary phase formation further, thus degraded the superelasticity of struts. Lastly, the laser power was the dominant indicator of the microstructural evolution in thin nitinol struts printing, whereas the energy density was not.

The Mₛ temperature of the SLM processed samples was generally higher than the powder (Mₛ: −17.9° C.). When printed at a low scanning speed of 50 mm/s and 150 mm/s, the Mₛ temperature of samples showed the same trend with respect to the increasing laser power from 50 W to 350 W. Specifically, the Mₛ temperature of the samples was kept at about −7.4° C. to 1.2° C. at the low scanning speed and low laser power region (50 W and 125 W). In contrast, it reached about 24.3° C. to 67.3° C. when the laser power was increased from 200 W to 280 W, respectively. This observation indicated that the Mₛ temperature met a threshold of transition at low scanning speed printing when the laser power was increased from 125 W to 280 W, corresponding to the energy density varied from 833 J/mm³ to 1867 J/mm³, respectively.

Accordingly, in some embodiments, the stent can have a temperature at which it initially transits from martensite to austenite (As (stent)) and a temperature at which it initially transits from austenite to martensite (Ms (stent)).

Correspondingly, the Nitinol powder has a temperature at which it initially transits from martensite to austenite (As (Nitinol powder)) and a temperature at which it initially transits from austenite to martensite (Ms (Nitinol powder)).

In some embodiments, As (stent) is lower than As (Nitinol powder) and Ms (stent) is higher than Ms (Nitinol powder).

In some embodiments, As (Nitinol powder) is about −6° and Ms (Nitinol powder) is about −18° C. In other embodiments, As is about −5.6° C. In other embodiments, Ms is about −17.9° C.

At the intermediate scanning speed of 500 mm/s, the Ms temperature was at about 23.2° C. and 23.7° C. when the laser power was at 50 W and 150 W; thereafter the Ms temperature increased to 33.4° C. and 33.9° C. when the laser power reached 200 W and 280 W. It jumped to about 60.5° C. when the laser power was increased to 320 W and 350 W. Herein, the threshold of transition in the Ms temperature occurred at a scanning speed of 500 mm/s combined with laser power of 280 W to 320 W, corresponding to the energy density of 187 J/mm³ to 213 J/mm³.

At the high scanning speed of 1500 mm/s, the Mₛ temperature slowly increased from 3.2° C. to 27.2° C. when the laser power was increased from 125 W to 350 W. No significant trend of transition was observed at the high scanning speed printing.

FIG. 16(a) shows the martensitic transformation starting temperatures (Mₛ) of nitinol stents printed using different laser power and scanning speed. FIG. 16(b) shows the process window for the Mₛ temperature distribution, where Region I represents T (Mₛ)<room temperature (RT); Region II represents RT<T(Mₛ)<body temperature (BT); Region III represents T(Mₛ)>BT, and Region IV represents failure in printing. FIG. 16(c) shows the energy density at different laser power and scanning speed.

X-ray Diffraction (XRD) spectra shows that the major peaks of austenitic phase (B2) and martensitic phase (B19′) were both shown when printed under P100 v100 and P200 v500. This was consistent with the phase transformation temperatures measured below RT. However, in the as printed P300 v1000 strut, a significant amount of peaks showing secondary phases, which were identified as the Ni₄Ti, Ti₂Ni, TiC, and NiCx, accordingly. By applying heat treatment on the stents, those secondary phases were disappeared from the XRD spectrum. The minor martensitic phase and secondary nickel-rich or titanium-rich phases could be attributed to the significant thermal instability in the high laser power combined with high scanning speed printing, and can be due to austenitic B2 phase dominated microstructures. Apparent noise and broadening at the base of the XRD peaks could have been a remnant from secondary phases with a low volume fraction.

The nitinol wt % of the SLM processed samples was further compared with the virgin nitinol powders. When printed at low laser power & low scanning speed (P100 v100), the energy density was 333.3 J/mm³. The nickel wt % varied from 54.64% to 55.63%. After heat treatment of newly printed samples, epitaxial columnar features showed similar nickel wt % with the surroundings, with nickel wt % varied from 54.66% to For the sample printed under P200 v500, the nickel wt % varied from 54.96% to 55.41% in the as printed state, and varied from 54.72% to 55.19% after heat treatment. The same XRD peaks in the as-printed and heat-treated struts further confirmed that the P200 v500 strut was dominated by the austenitic phase.

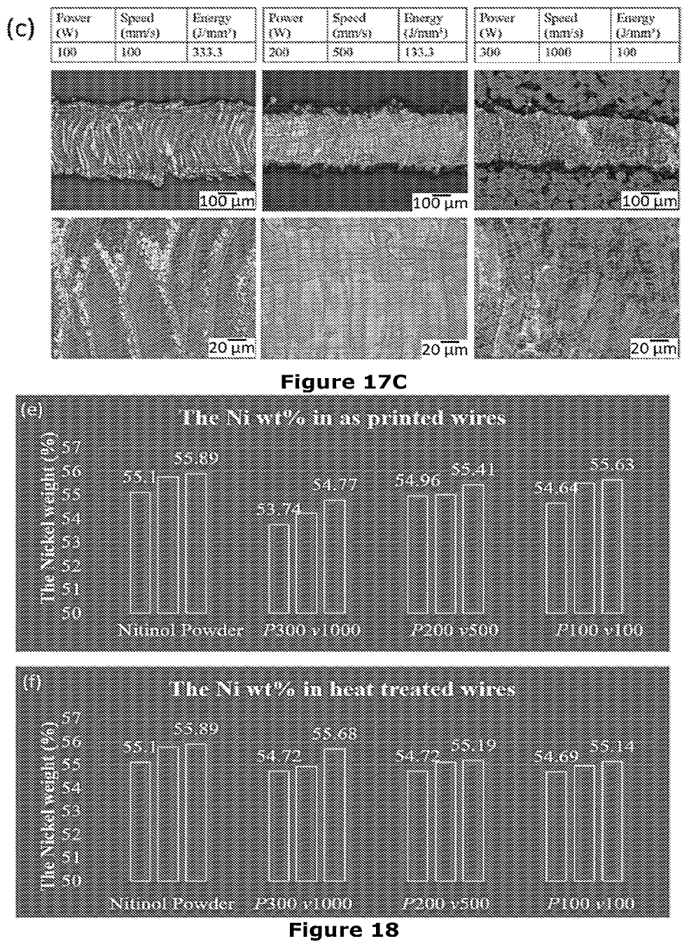

The strut printed at P300 v1000 represented the high laser power and high scanning speed combination, although the energy density was relatively low (100 J/mm³). The nickel wt % was at 53.74% to 54.77% in the as-printed state, whereas it has increased significantly after heat treatment, i.e., varied from 54.72% to 55.68%. The significant increase in the Ni wt % after heat treatment could originate from the dissolving of secondary Ni-rich phases. This was consistent with the XRD analysis of crystalline structures of samples. Apparently, as summarized in FIGS. 18a and 18b, after heat treatment, the Ni wt % of samples reached the same level when printed at P100 v100, P200 v500, and P300 v1000, suggesting similar levels of nickel evaporation occurred.

The above indicates that besides the volumetric energy density, the combination of SLM process parameters (i.e., scanning speed and laser power) also influenced the nickel weight percentage remarkably, and the phase transformation behavior of SLM fabricated nitinol alloys.

The printing process window for superelastic nitinol was indicated using the Ms temperature as an indicator summarized in FIG. 16. Four regions were classified corresponding to different ranges of Ms temperatures.

In Region I (FIG. 16b), the Ms temperature was scattered at around −7° C. to 4° C. The process window in Region I was defined as low power-low speed, i.e., 50 W<P<125 W and 50 mm/s<v<150 mm/s, accordingly. The energy density has ranged from 111.1 to 833.3 J/mm³, respectively. In Region I, the Ms temperature was increased by around 15° C. to 20° C. comparing to that of the virgin powders. The EDX analysis has shown a decrease in the nickel weight percentage by about 0.3% to 0.5% when printed using P100 v100 within this region. Moreover, the austenitic phase had dominated the strut P100 v100, both before and after the heat treatment has applied. This further suggested that superelastic nitinol stent could be printed using combined laser power and scanning speed in Region I, for applications at room temperature. Besides, printing parameters in Region I has caused minimal manipulation on the Ms temperature, referring to the powder used in this study.

In Region II (FIG. 16b), the Ms temperature was scattered around the room temperature (25° C.) to the body temperature (35° C.). The process window was defined as: (1) 50 W<P<125 W and v500 mm/s, (2) P200 W and 50 mm/s<v<1500 mm/s, (3) P280 W and 500 mm/s<v<1500 mm/s, and (4) 320 W<P<500 W and v1500 mm/s. The corresponding energy density has varied from 33.3 to 1333.3 J/mm³, respectively. In Region II, the Ms temperature was increased by about 40° C. to 50° C. compared to virgin powder. The significant increase in the Ms temperature could be attributed to nickel evaporation during the SLM process and nickel-rich secondary phase formation, or both. Two struts were printed using parameters in Region II, i.e., P200 v500 and P300 v1000, and the Ms temperatures were close to 35° C., respectively. The EDX results showed that the nickel weight percentage for P200 v500 was close to that of the P100 v100, either before or after heat treatment was applied. Moreover, the austenitic phase dominated the strut P200 v500, whereas minor peaks of the martensitic phase were eliminated by applying the heat treatment. In comparison, for the strut P300 v1000, a significant amount of Ni-rich secondary phase and martensitic phase was observed on the XRD peaks, corresponding to a lower level of Ni wt % detected using EDX comparing to the strut P100 v100 and P200 v500, respectively. Those secondary phases were eliminated, whereas the intensity of martensitic peaks was decreased by applying the heat treatment. Accordingly, the Ni wt % has been increased to similar levels of P100 v100 and P200 v500.

Two aspects could be suggested from this observation. Firstly, the level of nickel evaporation has kept consistent when printed using the parameters in Region I and Region II, i.e., decreased by about 0.3% to 0.5%. Secondly, increasing the laser power and scanning speed has facilitated the formation of Ni-rich secondary phases in the SLM process, which has further increased the Ms temperature of the struts printed. Lastly, those secondary phases could be eliminated using heat treatment. Therefore, the Ms temperature could be brought down using appropriate heat treatment conditions, accordingly.

Region III (FIG. 3b), where the Ms temperature has varied from around 50° C. to 70° C. when printed using the combination of high power & low speed, i.e., 280 W<P<350 W and 50 mm/s<v<500 mm/s, accordingly. The energy density has varied from 213 to 2333.3 J/mm³. In Region III, the Ms temperature was higher than that of the powder for about 80° C., which could be attributed to significant nickel evaporation due to the combination of high laser power and low scanning speed.

Furthermore, samples were peeled off from the substrate during the powder removing process when printed using parameters in Region IV (FIG. 3b), i.e., laser power below 125 W with a scanning speed of 1500 mm/s. The energy density was below 28 J/mm³, which was not sufficient for the full melting of nitinol powders.

Based on the stent design as disclosed herein, stents was printed with parameters P100 v100. The printed stents were with strut radius varied from 0.2093-0.2342 mm, and the overprint ratio was ranged from 39%-56% (overprint ratio=(printed diameter−designed diameter)/designed diameter) (FIG. 7c). The printing accuracy was relatively acceptable, with the strut diameter designed to be 0.3 mm. This overprint ratio can help to achieve the ideal diameters through precisely controlled material removal post-processing.

The correlation between laser power and speed is also shown in FIGS. 10A and 10B. Under Regime A, all of the Mₛ and Aₛ transformation temperatures of printed wires were below the temperature of 25° C. This was achieved when applying laser power at 50 W and 100 W combined with scanning speed of 50 mm/s, 100 mm/s and 150 mm/s, wherein the energy levels were varied from 111 to 667 J/mm³ (Regime A1-low power-low speed-high energy, also correlating to Region I of FIG. 16b). Similar decreased phase transformation temperature was observed when the laser power was increased to 150 W and 200 W while the scanning speed was increased to 500 mm/s, 1000 mm/s and 1500 mm/s, resulted energy density of 22 to 100 J/mm³ (Regime A2-high power-high speed-low energy), respectively. Table 4 tabulates the printing parameters.

Under the regime of A1, low laser power combined with low scanning speed resulted high levels of energy density, however the local temperature may maintained below certain threshold which has favoured the Ti-rich phase formation. Furthermore, Ni-rich secondary phase may also be formed during this process due to the complex thermal history involved in different sections of wire. Similarly, under the regime of A2, intermediate power but high speed may result in low local temperature which favoured secondary phase formation.

TABLE 4
Printed parameter combinations when under Regime A for printing
of vertical thin structures of diameter 0.3 mm and 1.5 cm height

| Sample | Power | Speed | Energy Density | Ms | As |
|---|---|---|---|---|---|
| A1-1 | 50 | 50 | 333.33 | 1.16 | −39.10 |
| A1-2 | 50 | 100 | 166.67 | 5.02 | −37.14 |
| A1-3 | 50 | 150 | 111.11 | 3.57 | −35.88 |
| A1-4 | 100 | 50 | 666.67 | −7.38 | −32.77 |
| A1-5 | 100 | 100 | 333.33 | −1.24 | −30.19 |
| A1-6 | 100 | 150 | 222.22 | −6.82 | −34.53 |
| A | 100 | 1500 | 22.22 | 2.42 | −39.02 |
| A2-1 | 150 | 500 | 100.00 | 14.15 | −29.32 |
| A2-2 | 150 | 1000 | 50.00 | −8.85 | −32.24 |
| A2-3 | 150 | 1500 | 33.33 | −7.81 | −28.38 |
| A2-4 | 200 | 1000 | 66.67 | 23.32 | −8.18 |
| A2-5 | 200 | 1500 | 44.44 | 19.91 | −8.45 |

Accordingly, the present invention provides a method of 3D printing a stent, comprising: performing selective laser melting on a Nitinol powder in order to form the stent, wherein selective laser melting is performed with:
i) a laser power of about 50 W to about 150 W, and a scanning speed of about 50 mm/s to about 1000 mm/s, or
ii) a laser power of about 150 W to about 250 W, and a scanning speed of about 500 mm/s to about 3000 mm/s.

In other embodiments, the selective laser melting is performed with a laser power of about 50 W to about 200 W, and a scanning speed of about 50 mm/s to about 150 mm/s. In other embodiments, the power is about 50 W to about 150 W, and a scanning speed of about 50 mm/s to about 900 mm/s, about 50 mm/s to about 800 mm/s, about mm/s to about 700 mm/s, about 50 mm/s to about 600 mm/s, about 50 mm/s to about 500 mm/s, about 50 mm/s to about 450 mm/s, about 50 mm/s to about 400 mm/s, about 50 mm/s to about 350 mm/s, about 50 mm/s to about 300 mm/s, about mm/s to about 250 mm/s, about 50 mm/s to about 200 mm/s, or about 50 mm/s to about 150 mm/s.

In some embodiments, the laser power is about 150 W to about 250 W, and the scanning speed is about 500 mm/s to about 2500 mm/s, about 500 mm/s to about 2000 mm/s, or about 500 mm/s to about 1500 mm/s.

FIG. 10A plots the laser power with respect to the Martensitic temperature of the stent. FIG. 10B plots the laser power with respect to the Austenitic temperature of the stent. For clarity, the data points are grouped according to the different Regimes. As shown in these plots, varying the laser power and scanning speed resulted in a 3D printed Nitinol stent with different groupings of Ms(stent) and As(stent).

In some embodiments, As (stent) is about −45° C. to about −25° C. In other embodiments, As (stent) is lower than As (Nitinol powder) but not below −45° C. In some embodiments, Ms (stent) is about −10° C. to about 10° C. These stents can, for example, be produced by Regime A1 of the method as disclosed herein.

In some embodiments, As (stent) is higher than As (Nitinol powder) and Ms (stent) is higher than Ms (Nitinol powder).

In some embodiments, As (stent) is about −30° C. to about 0° C. and Ms (stent) is about −10° C. to about 25° C. These stents can, for example, be produced by Regime A2 of the method as disclosed herein.

In some embodiments, the step of selective laser melting the Nitinol powder is such that the laser has a power of about 50 W to about 125 W, and a scanning speed is about mm/s to about 500 mm/s. This set of conditions falls within Regime A1 as disclosed above. In other embodiments, the power is about 50 W to about 120 W, about 50 W to about 110 W, or about 50 W to about 100 W. In other embodiments, the scanning speed is about 50 mm/s to about 450 mm/s, about 50 mm/s to about 400 mm/s, about 50 mm/s to about 350 mm/s, about 50 mm/s to about 300 mm/s, about 50 mm/s to about 250 mm/s, about 50 mm/s to about 200 mm/s, about 50 mm/s to about 150 mm/s, or about 50 mm/s to about 100 mm/s.

In some embodiments, the step of selective laser melting the Nitinol powder is such that the laser has a power of about 125 W to about 200 W, and a scanning speed of about 500 mm/s to about 1,500 mm/s. This set of conditions falls within Regime A2 as disclosed above. In other embodiments, the power is about 150 W to about 200 W, about 160 W to about 200 W, about 170 W to about 200 W, about 180 W to about 200 W, or about 190 W to about 200 W. In other embodiments, the scanning speed is about 600 mm/s to about 1500 mm/s, 700 mm/s to about 1500 mm/s, 800 mm/s to about 1500 mm/s, 900 mm/s to about 1500 mm/s, 1000 mm/s to about 1500 mm/s, 1100 mm/s to about 1500 mm/s, 1200 mm/s to about 1500 mm/s, 1300 mm/s to about 1500 mm/s, or 1400 mm/s to about 1500 mm/s.

Under regime B, all of the Mₛ and Aₛ transformation temperatures of printed wires were above the values of powders, when applying laser power at 50 W and 100 W combined with scanning speed of 500 mm/s, 1000 mm/s and 1500 mm/s, wherein the energy levels were varied from 11 to 67 J/mm³ (Regime B1-low power-high speed-low energy). Similar increased phase transformation temperature was observed when the laser power was increased to 150 W and 200 W while the scanning speed was decreased to 100 mm/s and 150 mm/s, resulted energy density of 133 to 1333 J/mm³ (Regime B2-high power-low speed-high energy), respectively. Furthermore, phase transformation temperatures were all above that of the powders when the laser energy was further increased to 280 W, 300 W, 320 W and 350 W, either under high speed of 500 mm/s to 1500 mm/s or under low speed of 50 mm/s to 150 mm/s, with an energy range from 62-233 J/mm³ for high speed and 622-2333 J/mm³ for low speed, respectively (Regime B3-high power-varies speed-varies energy, also correlating to Region II in FIG. 16b). Table 5 tabulates the printing parameters.

This could indicate a deficient of nickel in the printed wires. Firstly, regime B1 likely promote formation of nickel rich secondary phases such as Ni₃Ti during SLM process, thus the nickel element was decreased in the intermetallic nitinol phase. Secondly, when under high energy B2 and high power B3 regime, nickel will likely be evaporated during SLM process, caused an increase in the phase transformation temperature. This behaviour is even clear when comparing the B3 regime under high powder but low speed and high speed. When printed under the same high laser power, lower scanning speed resulted higher transformation temperatures than that of the higher scanning speed, further supporting nickel evaporation when under high energy levels.

TABLE 5
Printed parameter combinations when under Regime B for printing
of vertical thin structures of diameter 0.3 mm and 1.5 cm height

| Sample | Power | Speed | Energy Density | Ms | As |
|---|---|---|---|---|---|
| B1-1 | 50 | 500 | 33.33 | ≈100 | 23.69 |
| B1-2 | 50 | 1000 | 16.67 | ≈100 | 22.43 |
| B1-3 | 50 | 1500 | 11.11 | ≈100 | 22.87 |
| B1-4 | 100 | 500 | 66.67 | ≈100 | 23.19 |
| B2-1 | 150 | 50 | 1000.00 | 68.11 | 55.58 |
| B2-2 | 150 | 150 | 333.33 | 65.00 | 40.3 |
| B2-3 | 200 | 50 | 1333.33 | 87.27 | 24.37 |
| B2-4 | 200 | 100 | 666.67 | ≈100 | 23.35 |
| B2-5 | 200 | 150 | 444.44 | ≈100 | 26.77 |
| B2-6 | 200 | 500 | 133.33 | 33.42 | 35.43 |
| B3-1 | 280 | 500 | 186.67 | 33.91 | 31.38 |
| B3-2 | 280 | 1000 | 93.33 | 27.18 | 17.21 |
| B3-3 | 280 | 1500 | 62.22 | 24.93 | −6.16 |
| B3-4 | 300 | 500 | 200.00 | 56.92 | 43.62 |
| B3-5 | 300 | 1000 | 100.00 | 30.11 | 30.99 |
| B3-6 | 300 | 1500 | 66.67 | 25.89 | 21.86 |
| B3-7 | 320 | 500 | 213.33 | 60.80 | 49.15 |
| B3-8 | 320 | 1000 | 106.67 | 31.37 | 34.65 |
| B3-9 | 320 | 1500 | 71.11 | 27.19 | 30.48 |
| B3-10 | 350 | 500 | 233.33 | 60.36 | 51.76 |
| B3-11 | 350 | 1000 | 116.67 | 40.63 | 39.49 |
| B3-12 | 350 | 1500 | 77.78 | 26.23 | 30.16 |
| B3-13 | 280 | 50 | 1866.67 | 67.33 | 68.06 |
| B3-14 | 280 | 100 | 933.33 | 67.91 | 66.96 |
| B3-15 | 280 | 150 | 622.22 | 47.09 | 41.62 |
| B3-15 | 300 | 50 | 2000.00 | 64.24 | 71.78 |
| B3-17 | 300 | 100 | 1000.00 | 64.31 | 71.15 |
| B3-18 | 300 | 150 | 666.67 | 67.00 | 67.66 |
| B3-19 | 320 | 50 | 2133.33 | 64.58 | 56.97 |
| B3-20 | 320 | 100 | 1066.67 | 66.71 | 62.59 |
| B3-21 | 320 | 150 | 711.11 | 66.27 | 69.67 |
| B3-22 | 350 | 50 | 2333.33 | 64.68 | 51.62 |
| B3-23 | 350 | 100 | 1166.67 | 67.04 | 61.46 |
| B3-24 | 350 | 150 | 777.78 | 66.98 | 65.38 |

The above printing was performed on vertical thin structures of diameter 0.3 mm and 1 cm height for characterisation. It would be understood that printing of stent structures using these parameters could have slightly different results due to the angulation of the stent structures during printing.

The present invention provides a method of 3D printing a stent, comprising: performing selective laser melting on a Nitinol powder in order to form the stent, wherein selective laser melting is performed with:
iii) a laser power of about 150 W to about 250 W, and a scanning speed of about 50 mm/s to about 500 mm/s; or
iv) a laser power of about 250 W to about 350 W, and a scanning speed of about 500 mm/s to about 3000 mm/s.

In some embodiments, the selective laser melting is performed with a laser power of about 150 W to about 250 W, and a scanning speed of about 50 mm/s to about 450 mm/s, about 50 mm/s to about 400 mm/s, about 50 mm/s to about 350 mm/s, about mm/s to about 300 mm/s, about 50 mm/s to about 250 mm/s, about 50 mm/s to about 200 mm/s, or about 50 mm/s to about 150 mm/s.

In some embodiments, the selective laser melting is performed with a laser power of about 250 W to about 350 W, and a scanning speed of about 500 mm/s to about 2500 mm/s, about 500 mm/s to about 2000 mm/s, or about 500 mm/s to about 1500 mm/s.

In some embodiments, As (stent) is about 10° C. to about 25° C. and Ms (stent) is about C. to about 100° C. These stents can, for example, be produced by Regime B1 of the method as disclosed herein.

In some embodiments, As (stent) is about 10° C. to about 75° C. and Ms (stent) is about 60° C. to about 100° C. These stents can, for example, be produced by Regime B2 of the method as disclosed herein.

In some embodiments, As (stent) is about 10° C. to about 80° C. and Ms (stent) is about ° C. to about 70° C. These stents can, for example, be produced by Regime B3 of the method as disclosed herein.

In some embodiments, As (stent) is about 20° C. to about 75° C. and Ms (stent) is about 25° C. to about 100° C. These stents can, for example, be produced by Regime B of the method as disclosed herein.

In some embodiments, As (stent) is about −45° C. to about 80° C. and Ms (stent) is about −18° C. to about 100° C.

Taken altogether, the presently disclosed nitinol stent has a self-expanding deployment system, high flexibility and conformability due to a hybrid closed-cell design, thinner and rounder struts as well as adequate radial strength.

In some embodiments, the step of selective laser melting the Nitinol powder is such that the laser has a power of about 50 W to about 100 W, and a scanning speed of about 500 mm/s to about 1,500 mm/s. This set of conditions falls within Regime B1 as disclosed above. In other embodiments, the power is about 50 W to about 90 W, about 50 W to about 80 W, about 50 W to about 70 W, or about 50 W to about 60 W. In other embodiments, the scanning speed is about 600 mm/s to about 1500 mm/s, about 700 mm/s to about 1500 mm/s, about 800 mm/s to about 1500 mm/s, about 900 mm/s to about 1500 mm/s, about 1000 mm/s to about 1500 mm/s, about 1100 mm/s to about 1500 mm/s, or about 1200 mm/s to about 1500 mm/s.

In some embodiments, the step of selective laser melting the Nitinol powder is such that the laser has a power of about 150 W to about 200 W, and a scanning speed of about 50 mm/s to about 500 mm/s. This set of conditions falls within Regime B2 as disclosed above. In other embodiments, the power is about 160 W to about 200 W, about 170 W to about 200 W, or about 180 W to about 200 W. In other embodiments, the scanning speed is about 50 mm/s to about 450 mm/s, about 50 mm/s to about 400 mm/s, about 50 mm/s to about 350 mm/s, about 50 mm/s to about 300 mm/s, about 50 mm/s to about 250 mm/s, about 50 mm/s to about 200 mm/s, about 50 mm/s to about 150 mm/s, or about 50 mm/s to about 100 mm/s.

In some embodiments, the step of selective laser melting the Nitinol powder is such that the laser has a power of about 200 W to about 350 W, and a scanning speed of about 50 mm/s to about 1,500 mm/s. This set of conditions falls within Regime B3 as disclosed above. In other embodiments, the power is about 210 W to about 350 W, about 220 W to about 350 W, about 230 W to about 350 W, about 240 W to about 350 W, about 250 W to about 350 W, about 260 W to about 350 W, about 270 W to about 350 W, about 280 W to about 350 W, about 290 W to about 350 W, about 300 W to about 350 W, about 310 W to about 350 W, about 320 W to about 350 W, or about 330 W to about 350 W. In other embodiments, the scanning speed is about 60 mm/s to about 1500 mm/s, about mm/s to about 1500 mm/s, about 80 mm/s to about 1500 mm/s, about 90 mm/s to about 1500 mm/s, about 100 mm/s to about 1500 mm/s, about 200 mm/s to about 1500 mm/s, about 300 mm/s to about 1500 mm/s, about 400 mm/s to about 1500 mm/s, about 500 mm/s to about 1500 mm/s, about 600 mm/s to about 1500 mm/s, about 700 mm/s to about 1500 mm/s, about 800 mm/s to about 1500 mm/s, about 900 mm/s to about 1500 mm/s, about 1000 mm/s to about 1500 mm/s, about 1100 mm/s to about 1500 mm/s, about 1200 mm/s to about 1500 mm/s, or about 1300 mm/s to about 1500 mm/s.

Regimes A and B provide an indication of nickel-rich or titanium-rich precipitation phase formation conditions when varying the scanning speed and laser powers in SLM process. Advantageously, based on the above, after receiving the nitinol powders from a vendor, nitinol devices manufacturer can decide the regimes of parameters to refine based on the functional requirements of products, i.e. refine under Regime A to obtain products with lower phase transformation temperatures than the raw powders, while refine under Regime B to obtain products with higher phase transformation temperature than the raw powders. FIG. 10A-B demonstrates the distribution of printing parameters under different regimes.

In some embodiments, the selective laser melting is performed with a hatch distance of about 0.1 mm to about 0.5 mm. In other embodiments, the hatch distance is about 0.1 mm to about 0.4 mm, or about 0.1 mm to about 0.3 mm. In some embodiments, the selective laser melting is performed with a hatch distance of about 0.1 mm.

In some embodiments, the selective laser melting is performed with a layer thickness of about 0.01 mm to about 1 mm. In other embodiments, the layer thickness is about 0.01 mm to about 0.9 mm, about 0.01 mm to about 0.8 mm, about 0.01 mm to about 0.7 mm, about 0.01 mm to about 0.6 mm, about 0.01 mm to about 0.5 mm, about 0.01 mm to about 0.4 mm, about 0.01 mm to about 0.3 mm, about 0.01 mm to about 0.2 mm, about 0.01 mm to about 0.1 mm. In some embodiments, the selective laser melting is performed with a layer thickness of about 0.03 mm.

In some embodiments, a stent with a wire diameter of less than about 1 cm is 3D printed. In other embodiments, stent with a wire diameter of about 0.1 mm to about 0.9 mm is 3D printed, or about 0.1 mm to about 0.8 mm, about 0.1 mm to about 0.7 mm, about 0.1 mm to about 0.6 mm, about 0.1 mm to about 0.5 mm, about 0.1 mm to about 0.4 mm, about 0.1 mm to about 0.3 mm, or about 0.1 mm to about 0.2 mm.

For example, a stent with a wire diameter of about 0.1 mm can be printed using the following parameters:

| Laser power (W) | Scanning speed (mm/s) |
|---|---|
| 50 | about 50 mm/s to about 500 mm/s |
| 100 | about 50 mm/s to about 1500 mm/s |
| 150 | about 50 mm/s to about 1500 mm/s |
| 200 | about 50 mm/s to about 2000 mm/s |
| 250 | about 100 mm/s to about 2500 mm/s |
| 300 | about 500 mm/s to about 2500 mm/s |

For example, a stent with a wire diameter of about 0.2 mm to about 0.5 mm can be printed using the following parameters:

| Laser power (W) | Scanning speed (mm/s) |
|---|---|
| 50 | about 50 mm/s to about 1000 mm/s |
| 100 | about 50 mm/s to about 1000 mm/s |
| 150 | about 50 mm/s to about 2500 mm/s |
| 200 | about 50 mm/s to about 2500 mm/s |
| 250 | about 100 mm/s to about 2500 mm/s |
| 300 | about 500 mm/s to about 2500 mm/s |

In some embodiments, the 3D printed stent is characterised by a austenite finish temperature (A_{f}) of about 25° C. to about 50° C. In other embodiments, the A_{f} temperature is about 30° C. to about 50° C., about 30° C. to about 45° C., about 30° C. to about 40° C., or about 35° C. to about 40° C. In other embodiments, the A_{f} temperature is about 37° C.

In a preferred embodiment, the parameters are selected from:
a) when the laser power is less than about 100 W, the scanning speed is less than about 1000 mm/s;
b) when the laser power is about 100 W to less than about 200 W, the scanning speed is less than about 2000 mm/s;
c) when the laser power is about 200 W to less than about 250 W, the scanning speed is less than about 2500 mm/s;
d) when the laser power is about 250 W to less than about 300 W, the scanning speed is about 50 mm/s to less than about 3000 mm/s;
e) when the laser power is about 300 W to less than about 350 W, the scanning speed is about 100 mm/s to less than about 3000 mm/s.

The above parameters are suitable for stents with wire diameter of about 0.1 mm.

In a preferred embodiment, the parameters are selected from:
a) when the laser power is less than about 100 W, the scanning speed is less than about 1500 mm/s;
b) when the laser power is about 100 W to less than about 250 W, the scanning speed is less than about 3000 mm/s;
c) when the laser power is about 250 W to less than about 300 W, the scanning speed is about 50 mm/s to less than about 3000 mm/s;
d) when the laser power is about 300 W to less than about 350 W, the scanning speed is about 100 mm/s to less than about 3000 mm/s.

The above parameters are suitable for stents with wire diameter of about 0.2 mm.

In some embodiments, the method further comprises providing a template of the stent as disclosed herein.

The present invention also provides a 3D printed stent printed using the method as disclosed herein, the stent comprising Nitinol having a nickel content of about 54 wt % to about 57 wt % of the composition and a titanium content of about 43 wt % to about 46 wt % of the composition;
wherein the stent has a martensite to austenite transition (As) temperature of about −45° C. to about 80° C.; and
wherein the stent has a austenite to martensite transition (Ms) temperature of about −° C. to about 100° C.

In some embodiments, the stent has a martensite to austenite transition (As) temperature of about −45° C. to about 0° C. and a austenite to martensite transition (Ms) temperature of about −10° C. to about 25° C.

In some embodiments, when the selective laser melting is performed using the above conditions, the stent is characterised by columnar grains due to inter-layer over melt.

In some embodiments, the stent has a martensite to austenite transition (As) temperature of about 10° C. to about 80° C. and a austenite to martensite transition (Ms) temperature of about 20° C. to about 100° C.

In some embodiments, when the selective laser melting is performed using the above conditions, the stent is characterised by fully merged layer boundaries due to re-melt of an underlying layer.

In some embodiments, the 3D printed stent is characterised by a austenite finish temperature (A_{f}) of about 25° C. to about 50° C. In other embodiments, the A_{f} temperature is about 30° C. to about 50° C., about 30° C. to about 45° C., about 30° C. to about 40° C., or about 35° C. to about 40° C. In other embodiments, the A_{f} temperature is about 37° C.

In some embodiments, the nickel is about 54 wt % to about 56.9 wt %, about 54 wt % to about 56.8 wt %, about 54 wt % to about 56.7 wt %, about 54 wt % to about 56.6 wt %, about 54 wt % to about 56.5 wt %, about 54 wt % to about 56.4 wt %, about 54 wt % to about 56.3 wt %, about 54 wt % to about 56.2 wt %, about 54 wt % to about 56.1 wt %, about 54 wt % to about 56 wt %, about 54.1 wt % to about 56 wt %, about 54.2 wt % to about 56 wt %, about 54.3 wt % to about 56 wt %, about 54.4 wt % to about 56 wt %, about 54.5 wt % to about 56 wt %, about 54.6 wt % to about 56 wt %, about 54.7 wt % to about 56 wt %, about 54.8 wt % to about 56 wt %, about 54.9 wt % to about 56 wt %, about 55 wt % to about 56 wt %, about 55 wt % to about 55.9 wt %, about 55 wt % to about 55.8 wt %, about 55 wt % to about 55.7 wt %, about 55 wt % to about 55.6 wt %, or about 55 wt % to about 55.5 wt % of the composition. In other embodiments, nickel is about 54.5 wt % to about 55.8 wt % the composition. In some embodiments, the nickel is about 55.2 wt % the composition.

In some embodiments, the titanium is about 43 wt % to about 45.9 wt %, about 43 wt % to about 45.8 wt %, about 43 wt % to about 45.7 wt %, about 43 wt % to about 45.6 wt %, about 43 wt % to about 45.5 wt %, about 43 wt % to about 45.4 wt %, about 43 wt % to about 45.3 wt %, about 43 wt % to about 45.2 wt %, about 43 wt % to about 45.1 wt %, about 43 wt % to about 45 wt %, about 43.1 wt % to about 45 wt %, about 43.2 wt % to about 45 wt %, about 43.3 wt % to about 45 wt %, about 43.4 wt % to about 45 wt %, about 43.5 wt % to about 45 wt %, about 43.6 wt % to about 45 wt %, about 43.7 wt % to about 45 wt %, about 43.8 wt % to about 45 wt %, about 43.9 wt % to about 45 wt %, about 44 wt % to about 45 wt %, about 44.1 wt % to about 45 wt %, about 44.2 wt % to about 45 wt %, about 44.3 wt % to about 45 wt %, about 44.4 wt % to about 45 wt %, or about 44.5 wt % to about 45 wt % of the composition. In other embodiments, the titanium is about 44.5 wt % to about 45.2 wt % of the composition. In other embodiments, the titanium is about 44.8 wt % of the composition.

In some embodiments, wires of the 3D printed stent have a partially flat cross sectional shape. In some embodiments, wires of the 3D printed stent have an elliptical, tear drop, partially flattened tear drop or circular cross section shape.

In some embodiments, the 3D printed stent has a curvature along its longitudinal dimension when in the expanded state. In some embodiments, the 3D printed stent has a curvature of about 1° to about 160°. In some embodiments, the 3D printed stent has a radius of curvature of about 1 mm to about 200 cm.

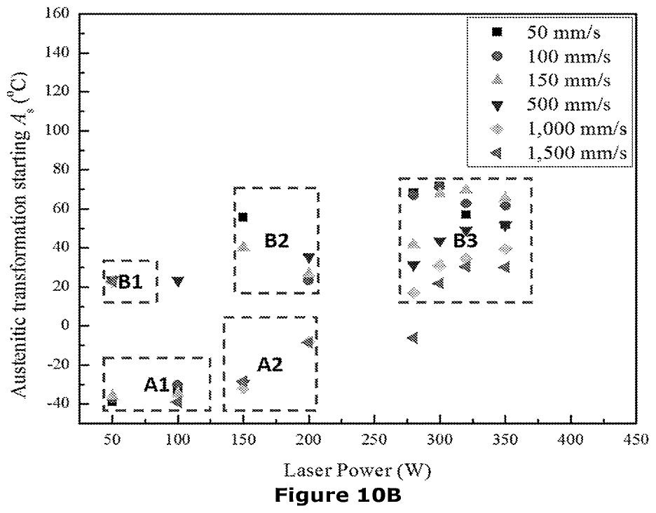

FIG. 11 illustrates the density of stents examined under High resolution X-ray Computed Tomography (HRXCT). When printed under Regime A1-low power-low speed-high energy, powders were fully melted thus the density of wires were high. When printed under Regime B1-low power-high speed-low energy, clear internal porous structures were shown. When printed under Regime B2-high power-high speed-high energy and Regime B3-high power-varies speed-varies energy, fully dense wires were obtained but the transformation temperatures were high due to either formation of Ni-rich phases or nickel evaporation caused by high energy density.

The 3D printed stent can have rough surfaces. To further improve the surface morphology, a post processing step can be added.

In some embodiments, the method further comprises a step of heat treating the stent.

In some embodiments, the heat treating step comprises heating the stent from about 200° C. to about 800° C. In other embodiments, the heating is from about 200° C. to about 750° C., about 200° C. to about 700° C., about 200° C. to about 650° C., about 200° C. to about 600° C., about 250° C. to about 600° C., about 300° C. to about 600° C., about 350° C. to about 600° C., about 400° C. to about 600° C., or about 450° C. to about 600° C. The heating step can be performed for about 10 min, about 20 min, about 30 min, about 40 min, about 60 min, or for more than 60 min.

In some embodiments, the method further comprises a step of heat treating the stent when the stent is printed using condition ii, iii or iv. In other embodiments, the method further comprises a step of heat treating the stent when the stent is printed outside condition i.

Based on the DSC results, 24 wire samples with Mₛ and A_{f} closer or below the room temperature was selected for mechanical tests, attempting to examine the superelasticity of those wires printed. Wire samples having diameters less than 1.0 mm and length of 15 mm, which were not suitable for the application of extensometer in the tensile tests using Instron equipment. A camera was used with digital image correlation and TEMA software to capture the accurate deformation strain with respect to load. The maximum stress (ultimate tensile stress) and fracture strain was summarized in the table below.

As shown in Table 6, under Regime A1-low power-low speed-high energy, the fracture strain has reached to 10%, indicating good ductility of the wire samples fabricated from SLM. However the fracture stress was low compared to other samples, likely due to high internal porosity since powders were not fully melted under Regime A1. Under Regime

B, the overall fracture strain was lower than that of regime A, however several samples under Regime B2-high power-low speed-high energy and Regime B3-high power-varies speed-varies energy showed acceptable fracture strain of about 8%-9%, most importantly high fracture stress was obtained under this regime.

TABLE 6
The maximum stress and strain at fracture
of the selected samples for tensile tests

| | | | Energy | Max | |
|---|---|---|---|---|---|
| | Power | Speed | Density | Stress | Max |
| Sample No. | (Watt) | (mm/s) | (J/mm³) | (MPa) | Strain |
| A1-1 | 50 | 50 | 333.33 | 224.927926 | 10.5% |
| A1-4 (close) | 125 | 50 | 833.33 | 534.494376 | 10.3% |
| B1-4 (close) | 125 | 500 | 83.33 | 630.573519 | 4.0% |
| B2-5 | 200 | 150 | 444.44 | 673.279832 | 8.0% |
| B2-6 | 200 | 500 | 133.33 | 657.164527 | 5.1% |
| B3-1 | 280 | 500 | 186.67 | 439.891747 | 11.0% |
| B3-3 | 280 | 1500 | 62.22 | 534.575239 | 0.91% |
| B3-7 | 320 | 500 | 213.33 | 750.144608 | 8.1% |
| B3-12 | 350 | 1500 | 77.78 | 667.969113 | 6.2% |
| B3-21 | 320 | 150 | 711.11 | 555.514803 | 9.0% |
| B3-24 | 350 | 150 | 777.78 | 512.404576 | 2.7% |

It was found that the structural geometry can be particularly advantageous for improving surface quality of fine Nitinol structures without changing process parameter.

Further, it was also found that the printing direction of the stent can be particularly advantageous for improving surface quality of fine Nitinol structures. When thin structures of diameter 0.3 mm were built at various inclination angles at 30°, 35°, 40°, 45° 50°, 55°, it was found that particles attachment was reduced in comparison to vertically printed structures. At the upskin region, at 30° inclination, it could be clearly observed the upskin section of the structure is free from particles. As the inclination angle increases, more particles attachment occurs at the upskin section but this increase was within a standard deviation.

In some embodiments, the method of 3D printing a stent comprises printing the stent such that the longitudinal dimension is at an angle to a horizontal plane of about 30° to about 60°. This can for example be done by providing the template of the stent with the longitudinal dimension at an angle, such that when read by the 3D printing machine, the stent is printable at an inclination angle to a horizontal plane of about 30° to about 60°. This advantageously reduces or eliminates balling effect on the stent on the upskin section.

It was found that a partially curved cross sectional morphology such as an arc provides for about a ten times reduction of downskin surface area roughness compared to a circular cross section.

The sharp edge of the partially curved cross-section (such as an arc-shape design) reduces the contact surfaces between the particles and the structure at the downskin section. With a reduction of contact surfaces between particles and structure, adherence of partially melted particles to the structure downskin will be weaker thus effort to remove the particles during post processing will be reduced.

Other partially curved geometry can also reduce particle bound to the stent structures. For example, arc, elliptical, tear drop or partially flattened tear drop (such as aerofoil) cross section shape can be used to further improve the surface finishing of the stent.

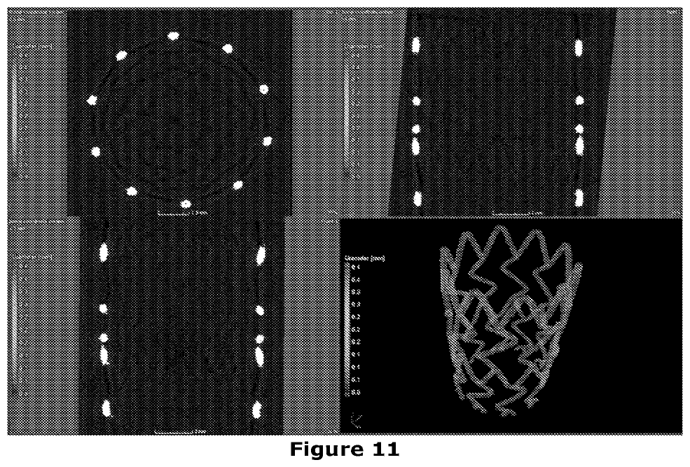

FIG. 12 shows examples of cross section shape that are particularly advantageous over a circular cross section shape. It was observed that less ballings were formed, which makes it easier to post process the 3D printed stent.

Reducing the structure thickness to the desired dimension can be achieved by surface treatment methods such as electropolishing. With the reduction of balling effect, surface finish after electropolishing can be enhanced.

The present invention also provides a method of 3D printing a stent, comprising:
a) providing a template of the stent; and
b) performing selective laser melting of a Nitinol powder based on the stent template in order to form the stent, wherein the stent template comprises:
i) at least two circumferential sections that are radially expandable in order for the stent to move from a collapsed state to an expanded state; and
ii) one or more flex sections, each flex section extending between two adjacent circumferential sections, each flex section being longitudinally expandable in order for the stent to move from the collapsed state to the expanded state;
wherein each flex section comprises a plurality of circumferentially arranged flex units, each flex unit comprising a wire having a wave-like structure; and
wherein in the expanded state, the flex unit forms an angle of about 15° to about 90° relative to a local radial plane at a junction with each of the adjacent circumferential sections; and
wherein selective laser melting is performed with either:
i) a laser power of about 50 W to about 150 W, and a scanning speed of about 50 mm/s to about 500 mm/s; or
ii) a laser power of about 150 W to about 250 W, and a scanning speed of about 500 mm/s to about 3000 mm/s;
iii) a laser power of about 150 W to about 250 W, and a scanning speed of about 50 mm/s to about 500 mm/s; or
iv) a laser power of about 250 W to about 350 W, and a scanning speed of about 500 mm/s to about 3000 mm/s.

The present invention also provides a stent delivery device, comprising:
a) a tube; and
b) a crimped stent slidably disposed within the tube, the crimped stent comprising Nitinol having a nickel content of about 54 wt % to about 57 wt % of the composition and a titanium content of about 43 wt % to about 46 wt % of the composition;
wherein the crimped stent has a martensite to austenite transition (As) temperature of about −45° C. to about 80° C.; and
wherein the crimped stent has a austenite to martensite transition (Ms) temperature of about −10° C. to about 100° C.;
wherein the crimped stent is adapted to revert back to its original uncrimped state when ejected from the tube and when exposed to a temperature of about 25° C. to about 50° C.

As the crimped stent has shape memory properties, the stent is revertible back to its original uncrimped state when at least exposed to a temperature above its Aₛ temperature.

The tube has a lumen for containing the crimped stent. The tube can be a catheter. In this regard, the tube can be a flexible tube of a suitable length and width.

The stent disposed within the tube is in a crimped state. In this regard, the stent is pressed or pinched into a small, compressed state. The stent holds itself in this state when the temperature is less than Mₛ temperature. The crimped stent has a smaller dimension compared to the lumen of the tube, and is thus slidable within the lumen of the tube. When the crimped stent is exposed to a temperature above Aₛ temperature, it reverts back to its original uncrimped state for deployment at the target site in the channel or vessel.

In some embodiments, the stent in its original uncrimped state is adapted to revert back to its original configuration after release of an external force and when exposed to a temperature of about 25° C. to about 50° C. This relies on the superelastic property of Nitinol processed according to the methods as disclosed herein.

In some embodiments, the stent delivery device further comprises ejecting means for ejecting the crimped stent out from the tube. The ejecting means can be a rod or wire insertable at one end of the tube for sliding the crimped stent out from the other end. The rod can be a flexible rod.

The present invention also provides a method of delivering a stent in a stent delivery device into a channel, the stent delivery device comprising:
a) a tube; and
b) a crimped stent slidably disposed within the tube, the crimped stent comprising Nitinol having a nickel content of about 54 wt % to about 57 wt % of the composition and a titanium content of about 43 wt % to about 46 wt % of the composition;
wherein the crimped stent has a martensite to austenite transition (Aₛ) temperature of about −45° C. to about 80° C.; and
wherein the crimped stent has a austenite to martensite transition (Mₛ) temperature of about −10° C. to about 100° C.;
wherein the crimped stent is adapted to revert back to its original uncrimped state when ejected from the tube and when exposed to a temperature of about 25° C. to about 50° C.;
the method comprising:
i) ejecting the crimped stent from the stent delivery device into the channel; and
ii) exposing the crimped stent to a temperature of about 25° C. to about 50° C. in order to revert the crimped stent to its original uncrimped state.

### Examples

### Simulation of Mechanical Properties of Flex Unit

To determine the flexibility and suitability of flex unit for 3D printing, different flex units were fabricated for comparison. These designs were compared with regard to the curvature and height of flex segments (FS), and how they fare against a cantilever beam that takes up the same width. With reference to FIG. 5A, all FS and the cantilever beam are 10 mm in width, and FS1 has 4 peaks with height (amplitude) 3 mm. FS2 has a height of 10 mm with 2 peaks, and FS3 is similar to FS2 except that it has a sharp peak. Both FS4 and FS5 have a height of 5 mm, and FS5 has a sharp peak compared to FS4. Similarly, both FS6 and FS7 have a height of 4 mm, and FS7 has a sharp peak compared to FS6.

Simulation studies of these models are performed with Autodesk Fusion 360, where each model is constrained on one end, with an arbitrary downward force of 1N placed on the other. Material selected is steel (Young's Modulus: 210000 MPa, Poisson's Ratio: 0.3, Yield Strength:207 MPa, Ultimate Tensile Strength: 345 MPa). FIG. 5A illustrates the stress concentration of each model, while FIG. 5B illustrates the amount of displacement. The deformation of each FS could also be observed from the simulations, where the faint black line denotes the original position of the FS.

### Simulation of Mechanical Properties of Stents

Simulations of designs 1 to 7 were carried out using Autodesk Fusion 360 to compare how each design performed when placed under conditions similar to the mechanical tests. The tests performed in the simulations are torsion, bending, axial tension and compression, with an equal arbitrary load of 10N for testing each different design for relative comparison. The material used for the simulations is steel, which is the same as what was used in the simulation of the flex segments. A base plate is added to the end of each model to facilitate the simulation, where the force will be placed on the base plate to simulate loading conditions.

Through the simulations, areas of the stent designs that will fail due to high stress and strain concentrations were identified. The results also revealed how different parameters such as number of circumferential units and strut unit thickness affects the performance of the stent, as well as how the different designs will deform when placed under different loading conditions.

Torsion: To simulate a torsional force on the various designs, the models are fully constrained on one end and a torsional force of 10N is applied onto the baseplate connected to the other end of the model. The baseplate is also constrained in the axis along the print direction (axis Y).

Bending: To simulate bending on the various designs, the models are fully constrained on one end and a vertical downward force of 10N is applied onto the baseplate connected to the other end of the model. The baseplate is also constrained in the axes that are not along the direction of the downward force (axes Y and Z).

Compression: To simulate compression on the various designs, the models are fully constrained on one end and a horizontal force of 10N along axis Y is applied onto the baseplate (towards the stent) connected to the other end of the model. The baseplate is also constrained in the axes that are not along the direction of the horizontal force (axes X and Z).

Tension: To simulate tension on the various designs, the models are fully constrained on one end and a horizontal force of 10N along axis Y is applied onto the baseplate (away from the stent) connected to the other end of the model. The baseplate is also constrained in the axes that are not along the direction of the horizontal force (axes X and Z).

### Transformation Temperatures—DSC

Differential scanning calorimeter (DSC) tests were conducted at heating/cooling rate of on the as-printed samples. The test temperature was ranged from −80° C. to 100° C. Several samples having peaks below −80° C. and above 100° C. were ignored. For the powders, the Mₛ temperature was at −18° C. while the Aₛ temperature was at −6° C.

We have purchased this composition to achieve superelasticity of wire after SLM printing.

### 3D Printing of Stent

The stent can be fabricated using a metal printer such as EOS M 290 3D printer, which has a fibre laser focus diameter of 100 μm, estimated laser affected area of 150-200 μm and estimated total affected area of 230-280 μm, as well as a building volume of 250×250×535 mm. The commercial Ni (55.4 wt %)-Ti powder (size rage 15-45 μm) was provided by Advanced Powders and Coating (GE Additive, Canada).

The laser power was varied from 50 W to 350 W. Meanwhile, the scanning speed was varied from 50 mm/s to 1,500 mm/s, contributed to an energy density ranged from 11.1 J/mm³ to 2,333.3 J/mm³ (Table 7). Moreover, the default hatch distance was at 0.1 mm, layer thickness was at 0.03 mm, and the oxygen level was controlled below 100 ppm.

In general, Nitinol struts having a diameter of 0.3 mm and height of 15 mm were designed and then printed vertically, reached an aspect ratio of 50:1. Thereafter, nitinol stents with a strut diameter of 0.3 mm were printed using the selected parameters.

TABLE 7
Exemplary list of energy density (J/mm³) corresponding
to different combinations of laser power (W)
and scanning speed (mm/s) applied.

| Energy | Scanning | Scanning | Scanning | Scanning |
|---|---|---|---|---|
| density | speed | speed | speed | speed |
| (J/mm³) | 50 mm/s | 150 mm/s | 500 mm/s | 1,500 mm/s |
| Power 50 W | 333.3 | 111.1 | 33.3 | 11.1 |
| Power 125 W | 833.3 | 277.7 | 83.3 | 27.7 |
| Power 200 W | 1333.3 | 444.4 | 133.3 | 44.4 |
| Power 280 W | 1866.6 | 622.2 | 186.6 | 62.2 |
| Power 320 W | 2133.3 | 711.1 | 213.3 | 71.1 |
| Power 350 W | 2333.3 | 777.7 | 233.3 | 77.7 |

Table 7 shows exemplary printing parameters that are suitable for printing stents with a strut (wire) diameter of about 0.1 mm to about 0.4 mm.

TABLE 7
Printing parameters of stents with strut diameter of about 0.1 mm to about 1 cm.

Speed (mm/s)

Circular strut Ø 0.1 mm

| | | | | | | | | |
|---|---|---|---|---|---|---|---|---|
| Power | 50 | 50* | 100* | 500* | 1000** | 1500** | 2000** | 2500** |
| (W) | 100 | 50* | 100* | 500* | 1000* | 1500* | 2000** | 2500** |
| | 150 | 50* | 100* | 500* | 1000* | 1500* | 2000** | 2500** |
| | 200 | 50* | 100* | 500* | 1000* | 1500* | 2000* | 2500** |
| | 250 | 50** | 100* | 500* | 1000* | 1500* | 2000* | 2500* |
| | 300 | 50** | 100** | 500* | 1000* | 1500* | 2000* | 2500* |

Circular strut Ø 0.2 mm

| | | | | | | | | |
|---|---|---|---|---|---|---|---|---|
| Power | 50 | 50* | 100* | 500* | 1000* | 1500** | 2000** | 2500** |
| (W) | 100 | 50* | 100* | 500* | 1000* | 1500** | 2000** | 2500** |
| | 150 | 50* | 100* | 500* | 1000* | 1500* | 2000* | 2500* |
| | 200 | 50* | 100* | 500* | 1000* | 1500* | 2000* | 2500* |
| | 250 | 50** | 100* | 500* | 1000* | 1500* | 2000* | 2500* |
| | 300 | 50** | 100** | 500* | 1000* | 1500* | 2000* | 2500* |

*Printable
**Not printable

### Sample Preparation and Material Characterisation

The 3D printed samples were mounted and ground by SiC 1200 grit sandpaper, and further polished with a grinding-polishing machine. The polished samples were etched for 180 seconds with ‘H₂O (82.7%), HNO₃ (14.1%), and HF (3.2%) solution’. The samples were cleaned with ethanol and pure water, then dried with an air gun.

Nikon Microscope (Nikon, Japan, ECLIPSE LV150N, S/N 251814) microscope under a bright field was used to examine the exposed microstructure. An energy dispersive X-ray detector ‘X-act’ (Oxford Instruments plc, United Kingdom), attached to a JOEL JSM-6010 PLUS/LV SEM (The Japan Electron Optics Laboratory Company, Limited, Japan), was used to examine the elemental composition on the exposed plane. The phases, or crystal structures, presented in those selected samples were accessed using Micro XRD (D8 Discover, Bruker, United States). Measurements were conducted at room temperature, with step intervals of 0.2° in 2θ ranged from 20° to 100°.

### X-Ray Computed Tomography

XCT was used to examine the three-dimensional geometric features of SLM processed nitinol stents. The GE Nanotom M (General Electric Company, United States) was used to capture thousands of images and reconstruct them into 3D volume. In addition, the VG studio Max 3.0 (Volume Graphics GmbH, Germany) was used for surface determination and volume analysis.

### Setup for Surface Quality Studies

Experiments were performed using a SLM printer Aconity3D MINI system with a 400 W Ytterbium Fiber Laser CW with a wavelength of 1070 nm. The laser spot is calibrated to the smallest achievable size of 55 μm. The chamber was purged with argon protection gas to keep the oxygen level below 100 ppm. A commercial Nitinol powder (NiTi) powder of 50-50 wt % (or about 55.4 wt % Ni) provided by the Advanced Powders and Coatings, GE Additive, was used as a raw material. The average powder size was about 15-53 μm.

Keyence VHX-6000 digital microscope was used to inspect the surface quality of the printed samples and measuring relative surface roughness profile of the polished samples. As printed samples for the metallographic tests were sliced, ground, polished, and then etched using a reagent (3 mL HCl, 1 mL HNO₃, and 96 mL H₂O) for 10 s. The microstructures were observed with an Olympus DM-4000M metallographic microscope (Wetzlar, Germany).

Printed specimens with different arc width underwent an electro-mechanical polishing process using the Dlyte machine. Particles attachment were absent with pit defects observed for all samples. Arc width of 0.1 mm has the least pit defect in the downskin section. The pit defects could be attributed to the partially fused particles attached to the downskin section. Pits were formed upon removed during electro-mechanical polishing.

It will be appreciated that many further modifications and permutations of various aspects of the described embodiments are possible. Accordingly, the described aspects are intended to embrace all such alterations, modifications, and variations that fall within the spirit and scope of the appended claims.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Throughout this specification and the claims which follow, unless the context requires otherwise, the phrase “consisting essentially of”, and variations such as “consists essentially of” will be understood to indicate that the recited element(s) is/are essential i.e. necessary elements of the invention. The phrase allows for the presence of other non-recited elements which do not materially affect the characteristics of the invention but excludes additional unspecified elements which would affect the basic and novel characteristics of the method defined.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A method of 3D printing a stent, comprising:
performing selective laser melting on a Nitinol powder in order to form the stent,
wherein selective laser melting is performed with:
i) a laser power of about 50 W to about 150 W, and a scanning speed of about 50 mm/s to about 1000 mm/s; or
ii) a laser power of about 150 W to about 250 W, and a scanning speed of about 500 mm/s to about 3000 mm/s; or
iii) a laser power of about 150 W to about 250 W, and a scanning speed of about 50 mm/s to about 500 mm/s; or
iv) a laser power of about 250 W to about 350 W, and a scanning speed of about 500 mm/s to about 3000 mm/s;
wherein when the selective laser melting is performed using conditions in (i), the 3D printed stent is characterised by a Aₛ temperature of about −45° C. to about −25° C.;
when the selective laser melting is performed using conditions in (ii), the 3D printed stent is characterised by a Aₛ temperature of about −30° C. to about 0° C.;
when the selective laser melting is performed using conditions in (iii), the 3D printed stent is characterised by a Aₛ temperature of about 10° C. to about 75° C.; and
when the selective laser melting is performed using conditions in (iv), the 3D printed stent is characterised by a Aₛ temperature of about 10° C. to about 80° C.

2. The method according to claim 1, wherein the selective laser melting is performed with:
i) a laser power of about 50 W to about 150 W, and a scanning speed of about 50 mm/s to about 500 mm/s; or
ii) a laser power of about 150 W to about 250 W, and a scanning speed of about 500 mm/s to about 1500 mm/s; or
iii) a laser power of about 150 W to about 250 W, and a scanning speed of about mm/s to about 150 mm/s; or
iv) a laser power of about 250 W to about 350 W, and a scanning speed of about 500 mm/s to about 1500 mm/s.

3. (canceled)

4. The method according to claim 1, wherein when the selective laser melting is performed using conditions in (i), the 3D printed stent is characterised by Mₛ temperature of about −10° C. to about 10° C.;
when the selective laser melting is performed using conditions in (ii), the 3D printed stent is characterised by a Mₛ temperature of about −10° C. to about 25° C.;
when the selective laser melting is performed using conditions in (iii), the 3D printed stent is characterised by a Mₛ temperature of about 60° C. to about 100° C.; and
when the selective laser melting is performed using conditions in (iv), the 3D printed stent is characterised by a Mₛ temperature of about 20° C. to about 70° C.

5. (canceled)

6. (canceled)

7. The method according to claim 1, wherein when the selective laser melting is performed using conditions in (i) or (ii), the 3D printed stent is characterised by columnar grains due to inter-layer over melt; and
wherein when the selective laser melting is performed using conditions in (iii) or (iv), the 3D printed stent is characterised by fully merged layer boundaries due to re-melt of an underlying layer.

8-12. (canceled)

13. The method according to claim 1, wherein the selective laser melting is performed with a hatch distance of about 0.1 mm to about 0.5 mm and/or a layer thickness of about 0.01 mm to about 1 mm.

14. (canceled)

15. The method according to claim 1, wherein the 3D printed stent has a wire diameter of less than 1 cm, preferably less than 0.5 mm.

16. The method according to claim 1, wherein the method further comprises a step of heat treating the stent from about 200° C. to about 800° C.

17. The method according to claim 1, wherein the method further comprises a step of heat treating the stent when the stent is printed using condition ii, iii or iv.

18. (canceled)

19. The method according to claim 1, wherein the 3D printed stent is characterised by a austenite finish temperature (A_{f}) of about 25° C. to about 50° C.

20. The method according to claim 1, wherein the 3D printed stent is characterised by wires of the 3D printed stent having a partially flat cross sectional shape, or by wires of the 3D printed stent having an elliptical, tear drop, partially flattened tear drop or circular cross section shape.

21. (canceled)

22. The method according to claim 1, wherein the 3D printed stent is characterised by a curvature along its longitudinal dimension when in the expanded state, preferably by a curvature of about 1° to about 160° and/or by a radius of curvature of about 1 mm to about 200 cm.

23. (canceled)

24. (canceled)

25. The method according to claim 1, wherein the method further comprises providing a template of the stent; wherein the stent template comprises:
i) at least two circumferential sections that are radially expandable in order for the stent to move from a collapsed state to an expanded state; and
ii) one or more flex sections, each flex section extending between two adjacent circumferential sections, each flex section being longitudinally expandable in order for the stent to move from the collapsed state to the expanded state;
wherein each flex section comprises a plurality of circumferentially arranged flex units, each flex unit comprising a wire having a wave-like structure; and
wherein in the expanded state, the flex unit forms an angle of about 15° to about 90° relative to a local radial plane at a junction with each of the adjacent circumferential sections.

26. The method according to claim 25, wherein the wave-like structure is a sinusoidal wave-like structure or a helical wave-like structure; wherein each flex unit has a wave number of about 0.5 unit to about 2 units; and/or wherein the wave-like structure in each flex unit has a peak characterised by an angle of about 15° to about 90° relative to a local radial plane at the peak.

27. (canceled)

28. (canceled)

29. The method according to claim 25, wherein when in the expanded state, each flex unit has a transverse breadth of about 2 mm to about 12 mm; and/or each flex unit has a longitudinal length of about 5 mm to about 15 mm.

30. (canceled)

31. The method according to claim 25, wherein a first end of at least one flex unit is connected to one of two adjacent circumferential sections by a first extension and/or a second end of at least one flex unit is connected to the other of the two adjacent circumferential sections by a second extension; wherein the first extension has a length of about 0.1 mm to about 5 mm and/or the second extension has a length of about 0.1 mm to about 5 mm.

32. (canceled)

33. A 3D printed stent comprising Nitinol having a nickel content of about 54 wt % to about 57 wt % of the composition and a titanium content of about 43 wt % to about 46 wt % of the composition;
wherein the stent has a martensite to austenite transition (As) temperature of about −° C. to about 80° C.;
wherein the stent has a austenite to martensite transition (Ms) temperature of about −° C. to about 100° C.,
wherein when the stent has a As temperature of about −45° C. to about 0° C. and a Ms temperature of about −10° C. to about 25° C., the stent is characterised by columnar grains due to inter-layer over melt; and
wherein when the stent has a As temperature of about 10° C. to about 80° C. and a Ms temperature of about 20° C. to about 100° C., the stent is characterised by fully merged layer boundaries due to re-melt of an underlying layer.

34. (canceled)

35. (canceled)

36. The 3D printed stent according to claim 33, wherein the 3D printed stent is characterised by a austenite finish temperature (A_{f}) of about ° C. to about 50° C.

37. The 3D printed stent according to claim 33, wherein the nickel is about 54.5 wt % to about 55.8 wt % the composition, preferably about wt % of the composition.

38. (canceled)

39. (canceled)

40. A stent delivery device, comprising:
a) a tube; and
b) a crimped stent slidably disposed within the tube, the crimped stent comprising Nitinol having a nickel content of about 54 wt % to about 57 wt % of the composition and a titanium content of about 43 wt % to about 46 wt % of the composition;
wherein the crimped stent has a martensite to austenite transition (As) temperature of about −45° C. to about 80° C.;
wherein the crimped stent has a austenite to martensite transition (Ms) temperature of about −10° C. to about 100° C.;
wherein when the stent has a As temperature of about −45° C. to about 0° C. and a Ms temperature of about −10° C. to about 25° C., the stent is characterised by columnar grains due to inter-layer over melt;
wherein when the stent has a As temperature of about 10° C. to about 80° C. and a Ms temperature of about 20° C. to about 100° C., the stent is characterised by fully merged layer boundaries due to re-melt of an underlying layer; and
wherein the crimped stent is adapted to revert back to its original uncrimped state when ejected from the tube and when exposed to a temperature of about 25° C. to about 50° C.

41-43. (canceled)

44. A 3D printed stent according to claim 33, wherein the 3D printed stent is characterised by a curvature along its longitudinal dimension when in the expanded state, preferably by a curvature of about 1° to about 160° and/or by a radius of curvature of about 1 mm to about 200 cm.
